(19) United States
(12) Patent Application Publication
(10) Pub. No.: US 2019/0169696 A1
(43) Pub. Date: Jun. 6, 2019

(54) Method of Detecting and/or Treating Colorectal Cancer Based on Divergent Liver Prometastatic Gene Expression Patterns

(71) Applicant: Persona Biomed, Inc., Alexandria, VA (US)

(72) Inventor: Fernando Vidal-Vanaclocha, Alexandria, MD (US)

(73) Assignee: Persona Biomed, Inc., Alexandria, VA (US)

(21) Appl. No.: 16/209,521

(22) Filed: Dec. 4, 2018

Related U.S. Application Data
(60) Provisional application No. 62/595,004, filed on Dec. 5, 2017.
(60) Provisional application No. 62/596,870, filed on Dec. 10, 2017.

(51) Int. Cl. C12Q 1/6886

(52) U.S. Cl. CPC C12Q 1/6886; C12Q 2600/118; C12Q 2600/158; G06F 19/24

(57) ABSTRACT

A method of detecting prometastatic cancer activity in the liver of a patient suffering from colorectal cancer, as well as a method of diagnosing and treating colorectal cancer according to primary tumor location, based on divergent expression levels and functional classification of specific genes in tumor unaffected hepatic tissue. Specific genes include statistically significant ones of group 1 genes PRDX4, CRP, ID1, MT1E, TNFSF14, MRC1, ICAM1, IL18, IL10, TFN; group 2 genes NGF, EPHA1, ERBB2IP, SDC1, COL18A1, KNG1, ADH1B, CYP2E1; and group 3 genes HP, VTN, RPS27, RPL23, GAPDH, TXN, VEGFA, CEACAM1, IGF1, TGFB1, DDR2, NOS2, and BMP7. These genes are compared with corresponding genes of individuals free of colorectal cancer in magnitude, correlation patterns, clustering patterns, heatmaps and other comparative analyses to derive insight into the tumor microenvironment to detect the onset of tumor growth, which enables early treatment well before conventional clinical signs emerge via any type of imaging, for example. Instead of analyzing the specifically denoted genes in hepatic tissue, the method may be implemented utilizing protein signatures of gene products derived from the patient's blood serum or plasma, in which case, the protein signatures indicate expression levels of the specific genes involved from which the analyses may be performed.

### CROSS-REFERENCE TO RELATED PATENT APPLICATIONS

This invention claims the benefit of U.S. Provisional Application Ser. No. 62/595,004 filed Dec. 5, 2017 in the name of the same inventor hereof, and entitled Method of Diagnosing and Treating Colorectal Cancer Based on Divergent Liver Prometastatic Gene Expression Patterns and U.S. Provisional Application Ser. No. 62/596,870 filed Dec. 10, 2017 in the name of the same inventor hereof and entitled Method of Processing Liver Prometastatic Gene Expression Patterns in a Rule-Based Diagnostic and Treatment Determination System, the disclosure of each of which is incorporated herein by reference.

### BACKGROUND

This invention concerns a method to detect metastatic cancer in a patient having colorectal cancer (CRC), as well as a method of diagnosing and treating such type of cancers. Specifically, the detection or diagnosis/treatment is made based on detection of divergent liver prometastatic gene expression patterns that occur in the tumor microenvironment well before the cancer has significantly developed or spread thereby providing an opportunity for early treatment and abatement of the disease. The invention is directed to use of protein signatures detected in a patient's blood serum or plasma to detect metastatic cancer cells in the liver.

The liver is a major metastasis-susceptible site in the human body and a majority of patients with hepatic metastases die from the disease regardless of treatment. Presently, hepatic metastasis is conventionally detected by imaging techniques, which typically cannot detect cancer lesions less than about five to seven millimeters in diameter. By the time the lesion reaches that size, millions or even billion of cancer cells have already spread throughout the patient's body and little if anything can be done to abate the disease. Thus, the average CRC patient dies within two to five years, more or less after clinical observation of hepatic metastasis.

A focal liver lesion in the liver, for example, more likely represents a metastatic tumor than a primary malignancy. In addition, a majority of CRC patients develop multiple liver metastases in both lobes that vary in diameter, suggesting that cancer cell seeding and growth occur in independent and separate episodes. Numerous experimental and clinical studies have focused on factors that regulate metastasis recurrence in the liver. At present, however, genetic and phenotypic properties of specific cancer cells able to implant and grow in the liver have not yet been established for any primary tumor type. Neither the contribution of the patient's genetic expressions nor the patient's physiologic background to the incidence and progression of hepatic metastases is presently understood.

Liver metastasis development is promoted by a broad range of organ-specific prometastatic factors, including cancer cell growth-stimulating factors, tumor stromal cell-stimulating factors, tumor angiogenesis-stimulating factors, and hepatic immune suppressant factors, among others. The experimental identification of some of these factors made it possible to understand certain hepatic metastasis development inhibition (Vidal-Vanaclocha F. The prometastatic microenvironment of the liver. Cancer Microenvironment, 2008, 1:113-129). However, it is not clear if these diverse factors have a control role during human liver metastasis disease. Neither is it clear if such factors have already occurred prior to CRC development (as a constitutive predisposition to liver metastasis), if they were induced by certain comorbidities and therapies, and/or if they were induced remotely by CRC cells endowed with this prometastatic feature.

Therefore, it is plausible that the liver might acquire a prometastatic condition concomitant with CRC progression and that such condition might be activated by either tumor-dependent or tumor-independent factors. Either way, these factors may activate remotely a “Liver Prometastatic Reaction” (LPR) favorable for the hepatic colonization of circulating cancer cells, and they should be designated as LPR-stimulating factors (LPR-SF), irrespective of their nature.

Production of LPR-SF and their delivery into the mesenteric vein circulation may be upregulated in CRC cells (including tumor and non-tumor stromal cell lineages) by tumor site-dependent factors (as for example, colonic inflammation, tumor hypoxia and mechanical stress, diet, gut microflora-derived bacterial factors, etc.), but also by factors from other intraperitoneal organs whose venous blood is draining into the mesenteric veins (spleen, pancreas, visceral fat, etc.). In addition, they may also be activated by systemic factors reaching the liver through the hepatic artery.

Once developed, the LPR may in turn lead to the hepatic cell production of Metastasis-Stimulating Factors (LPR-derived MSF) of potential interest as targets for anti-metastatic therapy. Their specific hepatic cell origin and their nature and effects on both cancer and stromal cells are now being recently understood. For example, LPR-derived MSF upregulated CRC cell expression of certain liver metastasis-specific genes, not expressed at primary CRC, suggesting they may also represent liver metastasis-specific molecular targets for therapy.

Therefore, LPR-specific genes and proteins may represent clinically-valuable hepatic biomarkers for predicting a risk level and/or detecting development of hepatic CRC metastasis. In addition, LPR-derived soluble factors should leave the liver through the suprahepatic vein and therefore they should be detectable in the peripheral blood, alerting on the occurrence of LPR in a given cancer patient.

The possibility that LPR-derived MSF can regulate some of liver metastasis-associated genes suggests that the CRC prometastatic phenotype includes both liver-independent and liver-dependent metastasis-associated genes, the first occurring at the primary tumor and the second only at metastatic sites, activated by the LPR-derived MSF. Therefore, liver-independent metastasis-associated CRC genes may have diagnostic value as prometastatic detectors or predictors at the primary tumor, while liver-dependent metastasis-associated CRC genes, which should be detectable at metastatic, but not at primary sites, may be valuable as targets for therapy. In addition, liver-independent metastasis-associated CRC genes may be involved in the CRC production of LPR-SF, which in turn would induce LPR-derived MSF further supporting hepatic metastasis development.

The inventor hereof has discovered that development of hepatic metastases is associated with an aberrant tissue-reconstitution process that results from bidirectional reciprocal effects between cancer cells and resident hepatic cells. On one hand, cancer cells and their soluble and exosomal proteins regulate gene expression in hepatic cells residing in, or infiltrating into, various sites of metastases. At these sites, cancer cells exert selective pressures on hepatic cells thereby shaping their functional phenotypes. Conversely, constituents of the liver microenvironment may also regulate gene expression in the cancer cells thereby controlling their fate and determining their ability to progress towards metastatic formation.

Additionally, there are pathophysiological processes such as aberrant hepatic regeneration, inflammation and fibrosis that change the hepatic microenvironment and notably affect development of metastases. Therefore, tumor microenvironment regulating hepatic metastasis in a given patient consists of structural and functional factors resulting from both hepatic-cancer cell interactions and previous or concurrent hepatic diseases.

Neoplasms from right and left colon and rectum frequently metastasize to the liver. At a transcriptional level, hepatic metastasis development is in part associated with marked changes in gene expression of colorectal cancer cells that may originate in a primary tumor. Other prometastatic changes occur in the liver and are regulated by hepatic cells, which represent a new microenvironment for metastatic colon cancer cells. In addition, hepatic parenchymal and non-parenchymal cell functions are also affected by both cancer cell-derived factors and various systemic pathophysiological factors of a patient having CRC.

Liver and gastrointestinal tract physiology and pathology are interrelated. For example, gallstones (cholelithiasis) and cholecystectomy are related to digestive system cancer through inflammation, altered bile flux, and changes in metabolic hormone levels. More importantly, it has been established that a statistically significant risk of colorectal cancer follows cholelithiasis (Lee et al, 2016; Gosavi et al, 2017). Similarly, fatty liver, which is a hepatic manifestation of metabolic syndrome, is a well-known risk factor for CRC (Barbois et al, 2017). If hepatic gene expression disorders precede CRC occurrence, early biomarkers of CRC risk and development may be assessed.

In the past two decades, a growing amount of data has been reported suggesting that carcinomas of the right and left colon should be considered as different tumor entities. Right-sided colon cancers (RCC) and left-sided colon cancers (LCC) are of different embryological origins, and various differences exist between them. Tumor location is associated with prognosis in colorectal cancer patients, and those with RCC have a significantly worse prognosis than those with LCC (Yahigi et al 2016). RCC should be treated distinctively from LCC (Zhao et al, 2017), and the establishment of standardized management for colon cancer by tumor location is needed.

Characterization of genes that are differentially expressed in tumorigenesis is an important step in identifying those that are intimately involved in the details of a cell's transformation from normal to cancerous, and from non-metastatic to metastatic cells. However, little is known about molecular changes that occur in key organs (as for example the liver) during the course of cancer development and its metastatic disease. While changes in the expression level of individual genes have been reported, investigation of gene expression changes that occur in the liver of patients with cancer and without cancer as provided by the present invention has not been previously known or documented.

In brief summary, there exists a need in the art for the identification of new CRC disease-associated hepatic genes and resultant proteins as molecular biomarkers to, among other things, to (i) monitor and assess the pathogenic contribution of liver to CRC and hepatic CRC metastasis development; (ii) identify and/or screen candidate cancer patients suitable for liver metastasis-specific therapies at the cancer microgenesis stage rather than conventional imaging techniques; and (iii) discover and/or screen pharmaceutical cellular and molecular compositions targeting those liver genes and gene products with CRC and CRC metastasis-stimulating activities in patients with colorectal cancer. These and other needs are met by various aspects of the present invention.

### SUMMARY

Proteins and enzymes are used to identify “liver prometastatic reaction level and class” in patients with and without CRC as part of a laboratory test to detect liver metastasis or to predict liver metastasis recurrence and the identification of candidate patients for liver metastasis-specific therapies. In addition, knowledge of metastatic CRC cell regulation by hepatic-derived prometastatic factors may provide opportunities for therapeutic intervention during CRC metastasis at both subclinical and advanced CRC stages.

A process is carried out to identity a transcriptional combination of liver-associated genes involved in inflammation, immune regulation, metabolic bioprotection and regeneration as a multiplex biomarker panel whose CRC-specific gene expression patterns define the “liver prometastatic reaction levels and classes” in patients with and without CRC and to alert of a possible occult CRC in patients with metabolic syndrome and cholelithiasis, as well as the possible location of such occult CRC in the left and right-sided colonic area and rectum. The process provides information on the specific status of liver genes involved in inflammation, immune regulation, metabolic bioprotection and regeneration, to help identify candidate patients for liver metastasis-specific therapies targeting identified genes, their associated functional mechanisms and their secondary disorders.

In accordance with a first aspect of the present invention, there is provided a method of detecting metastatic cancer in a target patient having a colorectal tumor, wherein the method comprises (a) obtaining a hepatic tissue sample from the target patient; (b) measuring by conventional means using standard techniques the genetic expression levels of a number of genes selected from group 1 genes (PRDX4, CRP, ID1, MT1E, TNFSF14, MRC1, ICAM1, IL18, IL10, TFN) and/or group 2 genes (NGF, EPHA1, ERBB2IP, SDC1, COL18A1, KNG1, ADH1B, CYP2E1); (c) comparing expression levels of genes measured in group 1 and/or group 2 with expression levels of respective genes indicative of a person free of a colorectal tumor; and (d) detecting that the target patient has metastatic cancer if certain ones of group 1 genes are overexpressed and/or certain ones of group 2 genes are underexpressed. The method may further include wherein the detecting step is carried out by detecting if a statistically significant number of group 1 and/or group 2 genes are respectively overexpressed or underexpressed or wherein the detecting step is carried out by detecting if statistically significant genes of group 1 and/or group 2 genes are respectively overexpressed and/or underexpressed. In accordance with another aspect of the invention, there is provided a method of detecting metastatic cancer in a patient having a colorectal tumor that comprises (a) obtaining a hepatic tissue sample from the patient, (b) measuring in said tissue sample genetic expression levels of multiple statistically significant genes selected from group 1 (PRDX4, CRP, ID1, MT1E, TNFSF14, MRC1, ICAM1, IL18, IL10, TFN), group 2 (NGF, EPHA1, ERBB2IP, SDC1, COL18A1, KNG1, ADH1B, CYP2E1) and/or group 3 (HP, VTN, RPS27, RPL23, GAPDH, TXN, VEGFA, CEACAM1, IGF1, TGFB1, DDR2, NOS2, and BMP7); (c) respectively comparing expression levels of selected genes of groups 1, 2 and 3 with expression levels of control genes indicative of individuals free of colorectal tumors; and (d) detecting if the target patent has metastatic cancer according to results of the comparing step.

In accordance with another aspect of the invention, there is provided a method of detecting metastatic cancer in a patient having a colorectal tumor wherein the method comprises (a) obtaining a hepatic tissue sample from the patient, (b) measuring genetic expression levels of multiple statistically significant genes of said tissue sample selected from group 1 (PRDX4, CRP, ID1, MT1E, TNFSF14, MRC1, ICAM1, IL18, IL10, TFN), group 2 (NGF, EPHA1, ERBB2IP, SDC1, COL18A1, KNG1, ADH1B, CYP2E1) and/or group 3 (HP, VTN, RPS27, RPL23, GAPDH, TXN, VEGFA, CEACAM1, IGF1, TGFB1, DDR2, NOS2, and BMP7); (c) respectively comparing expression levels of selected genes of groups 1, 2 and 3 with expression levels of control genes indicative of individuals free of colorectal tumors; (d) examining correlation and clustering patterns of gene expression levels measured in said group 1, group 2 and/or group 3 genes relative to expression levels of genes indicative of individuals free of colorectal tumors; and (e) detecting if the patient has metastatic cancer according to results of the examining.

In accordance with yet another aspect of the invention, there is provided a method of detecting occult cancer in a target patient having a gastrointestinal disorder (e.g., gallstones (cholelithiasis) and cholecystectomy) wherein the method comprises (a) obtaining a hepatic tissue sample from said patient; (b) in genes of said hepatic tissue sample, measuring expression levels of statistically significant ones of (i) metabolic bioprotection genes PRDX4, MT1E, CRP and NOS2, (ii) immune regulation genes ICAM1, IL10 and MRC1, and (iii) proinflammatory genes ID1, TNF-a, IL18 and TNFSF14 and/or statistically significant ones of (i) immune-regulation genes SDC1, COL18A1 and KNG1, (ii) proinflammatory genes EPHA1, CYP2E1, ADH1B, and (iii) fibrogenic/regeneration gene NGF; and (c) detecting occult cancer in said target patient if there are increased expression levels of statistically significant ones of (i) metabolic bioprotection genes PRDX4, MT1E, CRP and NOS2, (ii) immune regulation genes ICAM1, IL10 and MRC1, and (iii) proinflammatory genes ID1, TNF-a, IL18 and TNFSF14 and/or decreased expression levels of statistically significant ones of (i) immune-regulation genes SDC1, COL18A1 and KNG1, (ii) proinflammatory genes EPHA1, CYP2E1, ADH1B, and (iii) fibrogenic/regeneration gene NGF.

In accordance with yet a further aspect of the invention, there is provided a method of diagnosing and treating a patient suspected of having a subclinical liver micrometastasis disease or subclinical liver metastasis with a targeted gene therapy comprising (a) obtaining a hepatic tissue sample from the patient, (b) measuring in the hepatic tissue sample expression levels of genes from statistically significant ones of genes from group 1 (PRDX4, CRP, ID1, MT1E, TNFSF14, MRC1, ICAM1, IL18, IL10, TFN), group 2 (NGF, EPHA1, ERBB2IP, SDC1, COL18A1, KNG1, ADH1B, CYP2E1) and/or group 3 (HP, VTN, RPS27, RPL23, GAPDH, TXN, VEGFA, CEACAM1, IGF1, TGFB1, DDR2, NOS2, and BMP7), (c) comparing the measured expressions levels of genes in the previous step with expression levels of corresponding genes of persons known to be free of colorectal cancer, (d) segregating over-expressed and under-expressed gene expressions according to proinflammatory, immune regulation, metabolic protection and fibrogenic/regeneration classes of genes, and (e) treating said patient with anti-inflammatory therapy or immunotherapy according a statistically effective number of over-expressed and under-expressed genes residing in said respective classes. This method may further include treating a rectal tumor of said patient using immunotherapy with high-IL10, MRC1 and NOS2 gene expression; treating a left-sided colonic tumor of said patient using an anti-inflammatory therapy with high hepatic expressions of proinflammatory, immune regulation and metabolic bioprotection genes and decreased expression in BMP7 and NGF gene expressions; treating a right-sided colonic tumor of the patient using anti-inflammatory therapy with indication of a slight increase of proinflammatory and immune regulation gene expressions and decrease in ADH1B, SDC1 and VT gene expressions; or treating the patient by administering a drug that targets said selected liver prometastatic genes, as well as gene expression products and receptors thereof and associated signaling pathways thereof.

In accordance with yet a further aspect of the invention, there is provided a method detecting the anatomical location of an occult CRC tumor in a patient not having clinical evidence of CRC that comprises (a) obtaining a hepatic biopsy from the patient, (b) measuring in the biopsy expression levels of selected ones of prometastatic genes within proinflammatory, immune regulation, bioprotection and fibrogenic/regeneration functional classes of genes, (c) determining the identity of over-expressed and under-expressed ones of selected prometastatic genes within respective classes of genes, and (d) detecting the anatomical location of occult CRC in the patient according a statistically effective number of identified ones of over-expressed and under-expressed genes residing in the respective classes. This method may further include wherein the detecting step comprises detecting a rectal location of the CRC tumor according to underexpressed levels of statistically significant ones of (i) IL18, ID1, VEGFA, TNFSF14, ADH1B and CYP2E1 proinflammatory genes, (ii) ICAM1, KNG1, SDC1 AND BMP7 immuno regulation genes, and (iii) GAPDH, TXN, MTE1, HP, CR AND ERBB2IP metabolic bioprotection genes; wherein said detecting step comprises detecting a left-side colon location of the CRC tumor according to overexpressed levels of statistically significant ones of (i) proinflammatory genes IL18, ID1, TNF, TNFSF14, AND ADH1B, (ii) immune regulation genes ICAM1, MRC1, KNG1, and SDC1, and (iii) metabolic bioprotection genes PRXD4, MTE1, P, NOS2 and CRP; or wherein said detecting step comprises detecting right side colon location of the CRC tumor according to (i) a high expression level of at least one of ID1 and TNF proinflammatory genes, (ii) a low expression level of at least one of ADH18 and CYPE1 proinflammatory genes, (iii) a high expression level of at least one of immune regulation genes IL10, MRC1 and BMP7, (iv) low expression level of at least one of immune regulation genes KNG1 and SDC1, and (v) a low expression level of at least one of VTN and NGF fibrogenic and regeneration genes.

Yet, a further aspect of the invention comprises a method of colorectal cancer (CRC) screening using hepatic tissue biopsies sampled by the most appropriate procedure (e.g., percutaneous, transjugular, laparoscopic, directly during abdominal surgery) before, during or after open intraperitoneal surgery in patients with cholelithiasis (gallstones) and obesity (metabolic syndrome) and high CRC risk factors (as a complementary diagnostic test) wherein the method comprises measuring the expression level of liver prometastatic genes from group 1 genes (PRDX4, CRP, ID1, MT1E, TNFSF14, MRC1, ICAM1, IL18, IL10, TFN), group 2 (NGF, EPHA1, ERBB2IP, SDC1, COL18A1, KNG1, ADH1B, CYP2E1); and/or group 3 (HP, VTN, RPS27, RPL23, GAPDH, TXN, VEGFA, CEACAM1, IGF1, TGFB1, DDR2, NOS2, and BMP7), inhepatic tissue biopsies sampled during the surgical treatment of cholelithiasis and the bariatric surgery of patients with obesity; wherein the CRC-specific expression, correlation and clustering patterns of these liver prometastatic genes are indicative of occult CRC in those patients with no previous clinical evidence of CRC.

A further aspect of the invention includes a method of detecting a high CRC risk probability in a target patient having cholelithiasis (gallstones) and/or obesity (metabolic syndrome) comprising the steps of: (a) detecting expression levels of liver prometastatic genes from a statistically effective number of genes selected from group 1 (PRDX4, CRP, ID1, MT1E, TNFSF14, MRC1, ICAM1, IL18, IL10, TFN); group 2 (NGF, EPHA1, ERBB2IP, SDC1, COL18A1, KNG1, ADH1B, CYP2E1) and/or group 3 (HP, VTN, RPS27, RPL23, GAPDH, TXN, VEGFA, CEACAM1, IGF1, TGFB1, DDR2, NOS2, and BMP7) in hepatic tissue biopsies sampled for example by conventional procedures (e.g., percutaneous, transjugular, laparoscopic, directly during abdominal surgery) before, during and/or after surgical treatment of cholelithiasis and/or bariatric surgery wherein CRC-specific expression, correlation and clustering patterns of liver prometastatic genes as indicated in Tables 1-6 and FIGS. 3-13 hereof (including overexpression of PRDX4, MT1E, TNFSF14, MRC1, ICAM1, IL18, IL10, TNF, ID1 and CRP genes and underexpression of NGF, EPHA1, ERBB2IP, SDC1, COL18A1, KNG1, ALDH1B, CYP2E1 genes; (b) comparing expressions levels measured in the previous step with expression levels of corresponding genes of patients known to be free of CRC risk factors to detect new correlations of expression levels among metabolic bioprotection genes and among proinflammatory and metabolic bioprotection genes and loss of expression correlation among proinflammatory-fibrogenic/regeneration and immune regulation genes; wherein new gene clustering patterns for PRDX4, SDC1, VEGFA, ID1 and CRP genes indicate occult CRC in patients having no previous clinical evidence of CRC, and (c) detecting high CRC risk factors in the target patients according to upregulated and downregulated gene expressions detected in the previous step.

A further aspect of the invention comprises a method of detecting in a target patient subclinical liver micrometastasis disease or liver metastasis high-propensity and susceptibility comprising the steps of (a) measuring expression levels of certain liver prometastatic genes in hepatic tissue biopsies sampled for example by conventional procedures (percutaneous, transjugular, laparoscopic, directly during abdominal surgery) and (b) detecting subclinical liver micrometastasis disease or liver metastasis high-propensity and susceptibility according to CRC-specific expression, correlation and clustering patterns of liver prometastatic genes by detecting, in comparison with correlation patterns of persons free of CRC risks (i) overexpression of PRDX4, MT1E, TNFSF14, MRC1, ICAM1, IL18, IL10, TNF, ID1 and CRP genes (ii) underexpression of NGF, EPHA1, ERBB2IP, SDC1, COL18A1, KNG1, ALDH1B, CYP2E1 genes; (iii) altered correlation pattern of expressions among metabolic bioprotection genes and among proinflammatory and metabolic bioprotection genes; (iv) loss of expression correlation among proinflammatory-fibrogenic/regeneration and immune regulation genes; and (v) new gene clustering pattern for PRDX4, SDC1, VEGFA, ID1 and CRP genes).

Another aspect of the invention comprises a method of detecting CRC (i.e., liver prometastatic reaction level and class thereof) in a target patient with or without CRC comprising: (a) obtaining a hepatic tissue sample, (b) detecting in the sample expression levels of prometastatic genes from a statistically effective number of genes selected from group 1 (PRDX4, CRP, ID1, MT1E, TNFSF14, MRC1, ICAM1, IL18, IL10, TFN); group 2 (NGF, EPHA1, ERBB2IP, SDC1, COL18A1, KNG1, ADH1B, CYP2E1) and/or group 3 (HP, VTN, RPS27, RPL23, GAPDH, TXN, VEGFA, CEACAM1, IGF1, TGFB1, DDR2, NOS2, and BMP7), (c) comparing expression levels, correlation and/or clustering patterns of group 1, group 2 and group 3 liver prometastatic genes (i.e., in comparison with individuals free of CRC risk) by detecting (i) overexpression of selected ones of PRDX4, MT1E, TNFSF14, MRC1, ICAM1, IL18, IL10, TNF, ID1 and CRP genes; (ii) underexpression of selected ones of NGF, EPHA1, ERBB2IP, SDC1, COL18A1, KNG1, ALDH1B, CYP2E1 genes; (iii) new expression correlation among metabolic bioprotection genes and among proinflammatory and metabolic bioprotection genes; (iv) loss of expression correlation among proinflammatory-fibrogenic/regeneration and immune regulation genes; and (v) new gene clustering pattern for selected ones of PRDX4, SDC1, VEGFA, ID1 and CRP genes), and (c) detecting CRC risk in the target patient according to the previous step whereby to indicate subclinical liver micrometastasis disease or liver metastasis high-propensity and susceptibility.

Another aspect of the invention comprises a method of diagnosing and treating a patient with CRC comprising (a) obtaining from the patient a sample of hepatic tissue or blood serum/plasma, (b) sad sample, measuring expression levels of liver prometastatic genes or proteins to identify abnormal genes or gene activity (i.e., protein production) being overexpressed and underexpressed, and (c) treating the patient with a liver metastasis-specific therapy that targets (i) said abnormal genes, (ii) specific gene expression products and receptors of said abnormal genes and/or (iii) associated signaling pathways of said abnormal genes.

Another aspect of the invention comprises a method of detecting liver metastasis or risk thereof in a patient afflicted with CRC, obesity, gallstones, or any other disease increasing CRC risk, said method comprising the steps of (a) obtaining from the patient a sample of blood serum or plasma to be examined; (b) determining a protein signature of the sample by measuring the presence and/or amount of two or more proteins encoded by the genes of group 1 (PRDX4, CRP, ID1, MT1E, TNFSF14, MRC1, ICAM1, IL18, IL10, TFN) and/or group 2 (NGF, EPHA1, ERBB2IP, SDC1, COL18A1, KNG1, ADH1B, CYP2E1) and/or group 3 (HP, VTN, RPS27, RPL23, GAPDH, TXN, VEGFA, CEACAM1, IGF1, TGFB1, DDR2, NOS2, and BMP7), and (c) detecting liver metastasis if the presence and/or amount of the two or more proteins differs from a baseline protein signature of a normal, or healthy, individual not suffering from CRC, obesity, gallstones, or any other disease increasing CRC risk. Further aspect of the invention comprises a method according to the preceding steps to detect beneficial effects of treatment (i.e., by administering a therapeutic agent) of the patient, further comprising a step (d) of repeating steps (a), (b) and (c) to assess reduction in differences between the protein signatures whereby to indicate whether the therapeutic agent has a beneficial effect. By “protein signature” it is meant to include a combination of the presence and/or amount of a plurality of proteins present in a serum/plasma sample from an individual having CRC, obesity, gallstones, and any other disease increasing CRC risk, which protein combination can be distinguished from a combination of the presence and/or amount of proteins present in a serum/plasma sample from a normal, or healthy, individual not suffering from CRC, obesity, gallstones, and any other disease increasing CRC risk.

The above and further aspects of the invention will become apparent upon review of the following description taken in connection with the accompanying drawings. The invention, though, is pointed out by the appended claims.

### BRIEF DESCRIPTION OF THE DRAWINGS

FIGS. 1A and 1B show comparative transcriptomic analysis among metastatic CRC tissue, tumor-unaffected hepatic tissue and peripheral blood mononuclear cells from stage IV patients with CRC having systemic metastasis disease where FIG. 1A illustrates hybridization between RNA from metastatic CRC tissue and tumor-unaffected hepatic tissue and FIG. 1B illustrates hybridization between RNA from metastatic CRC tissue and peripheral blood mononuclear cells.

FIG. 1C shows a combination of FIGS. 1A and 1B in a single analysis diagram,

FIG. 1D is a Venn diagram showing overlapping sets of the number of genes for each of the three samples where CMN indicates mononuclear cells, M represent metastatic cells, and H represents tumor unaffected hepatic tissue.

FIG. 2 shows a logarithmic scale representation of the relative quantification (RQ) values of the liver prometastatic gene expression in CRC patients with respect to same values in patients without CRC.

FIG. 3 shows divergent liver prometastatic gene expression patterns in patients with and without CRC.

FIGS. 4A-4D respectively show distribution of liver prometastatic gene high-expressing patients by functional categories (proinflammatory, immune-regulation, metabolic Bioprotection and fibrogenic/regeneration) and tumor location in patients with CRC.

FIGS. 5A-5H show comparisons between proinflammatory gene expression levels in liver from patients with and without CRC.

FIGS. 6A-6I show comparisons between immuno-regulation gene expression levels in liver from patients with and without CRC.

FIGS. 7A-7H show comparisons between metabolic bioprotection gene expression levels in liver from patients with and without CRC.

FIGS. 8A-8F show comparisons between Fibrogenic and Regeneration gene expression levels in liver from patients with and without CRC.

FIG. 9 show comparison of overall expression profiles across samples from patients with and without CRC via a principal component analyses (PCA) of gene expressions in patients (P1-P51) with and in patients (C52-C72) without CRC where a first principal component (Dim 1) reveals a 27.18% of the variation and a second (Dim 2) reveals a 15.65% variation that separated most of patients with CRC from patients without CRC.

FIGS. 10A and 10B show Partial Least Squares-Discriminant Analysis (PLS-DA) intended to discriminate patients with and without CRC based on their hepatic expression level of liver prometastatic genes where elliptical shapes of FIG. 10A adopted by lines define the position coordinates of included patients (C indicates patients without CRC and P indicates patients with CRC). In this case, the discriminatory capacity was associated with the first component in the analysis. In FIG. 10B, position coordinates of liver prometastatic genes are plotted in correlation circles whose diameters define the influence of genes in the prediction of the class of patient where in this case metabolic bioprotection and fibrogenic/regeneration genes are in the smaller circle indicating that their expression levels had less ability to predict the patient's class than immune protection and proinflammatory genes, mainly located in the large correlation circle, and therefore had a greater predictive capacity to discriminate patients with and without CRC.

FIGS. 11A and 11B show heatmaps of clustering data for patients with and without CRC according their liver prometastatic gene expression patterns based on ΔΔCt ratios.

FIG. 12 shows Spearman's correlation patterns among liver prometastatic genes in patients with and without RCC.

FIGS. 13A-13D show hierarchical clustering was performed based on Pearson's correlation of Euclidean distance among the genes and gene clusters, and the results presented as a dendrogram plot in order to define the transcriptional structure of prometastatic genes in hepatic biopsies from patients without and with CRC (FIGS. 13A and 13C) and where a cluster including PRX4, SDC1, VEGFA, ID1 and CRP genes defined a main change in the hepatic transcriptional structure between patients without and with CRC (FIGS. 13B and 13D).

### DESCRIPTION OF ILLUSTRATIVE EMBODIMENTS

Disclosed herein are analytical procedures to detect occult CRC and liver metastasis risk and recurrence (i.e., a complementary testing and diagnostic procedure) and a method to identify candidate patients reasonably suitable to receive liver metastasis-specific therapies (a companion diagnostic test). An aspect of the invention uses, among other things, a series of mathematical, correlation and statistical analysis techniques to examine, compare and analyze relationships between and among expression levels of uniquely identified genes of hepatic tissues from patients with and without CRC. Thus, the invention may but not necessarily include utilization of a data processing device to automate gene analyses presented herein in order to provide a computer-determined output or result for diagnostic and/or treatment guidance to health care practitioners.

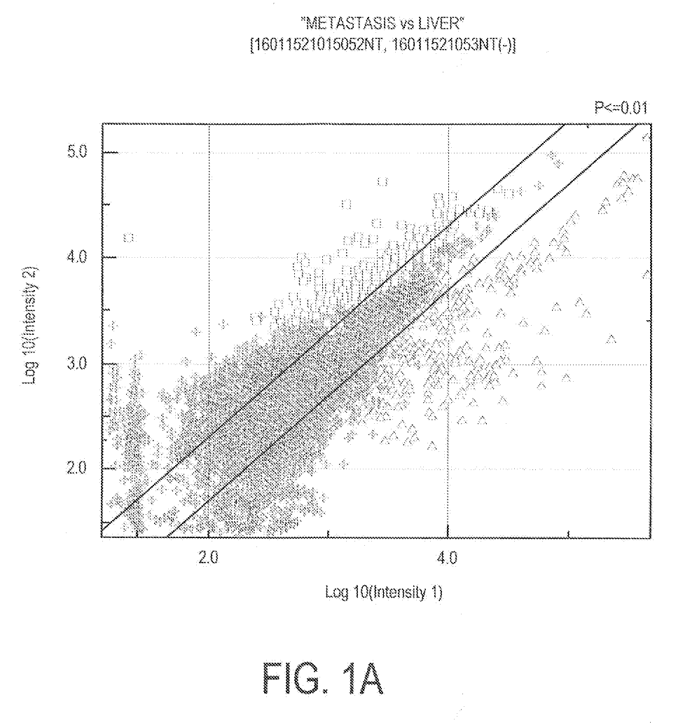

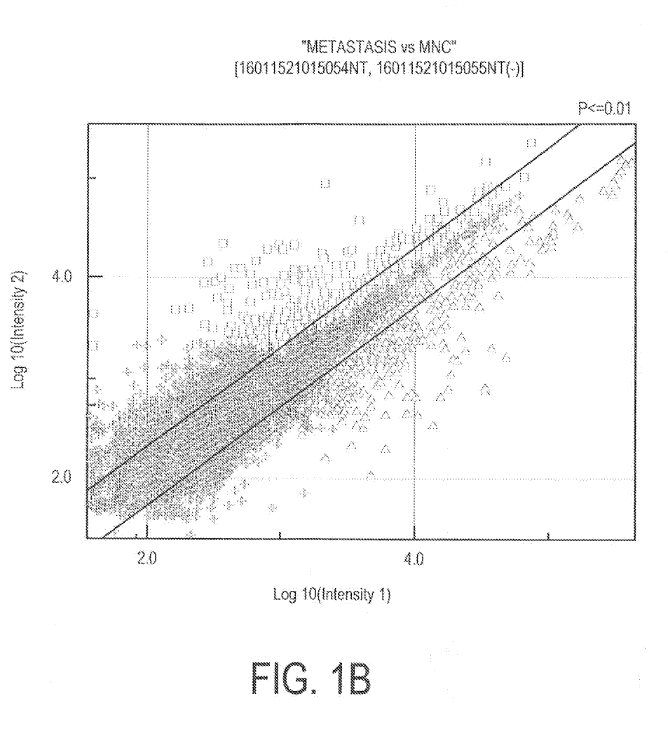

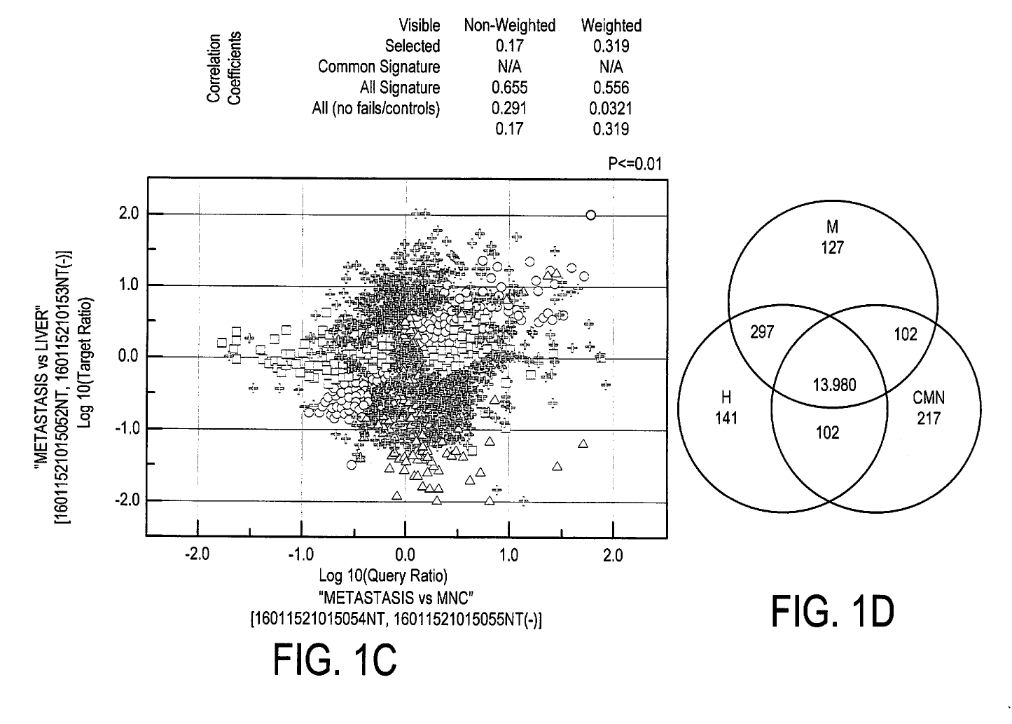

FIG. 1A shows a comparative transcriptomic analysis between metastatic CRC tissue and tumor-unaffected hepatic tissue from Stage IV patients with CRC having systemic metastasis disease. FIG. 1B shows a comparative transcriptomic analysis between metastatic CRC tissue and peripheral blood mononuclear cells from Stage IV patients with CRC having systemic metastasis disease. FIG. 1C combines that results of FIGS. 1A and 1B, while FIG. 1D shows the data in set and subset relation. More specifically, FIG. 1A shows hybridization between RNA from Metastatic CRC tissue and tumor-unaffected hepatic tissue; FIG. 1B shows hybridization between RNA from Metastatic CRC tissue and peripheral blood mononuclear cells; FIG. 1C shows a combination of FIGS. 1A and 1B; and FIG. 1D shows the number of genes for each sample compared and illustrated using a Venn diagram where “H” represents hepatic tissue, “M” represents metastatic tissue, and “CMN” represents mononuclear blood cells.

According to the analysis described in connection with FIGS. 1A, 1B, 1C and 1D, Table 1 below lists 122 genes whose expression levels were more than two-fold-upregulated in tumor-unaffected hepatic tissue compared to the expression levels in metastatic tissue and peripheral blood mononuclear cells from Stage IV patients with CRC having systemic metastasis disease. In bold are twenty-one genes whose expression levels were upregulated in liver parenchymal and non-parenchymal sinusoidal cells given the conditioned medium from cultured CRC cells (HT-29 CRC cell line). This gene subset was selected for further analysis.

TABLE 1

| | Upregulated Genes |
|---|---|
| A1BG | Alpha-1-B glycoprotein |
| A2M | Alpha-2-macroglobulin |
| ABAT | 4-aminobutyrate aminotransferase |
| ACAA2 | Acetyl-Coenzyme A acyltransferase |
| ACAT1 | Acetyl-Coenzyme A acetyltransferase 1 |
| | (acetoacetyl Coenzyme A thiolase) |
| ADH1A | Alcohol dehydrogenase 1A (class I), alpha polypeptide |
| ADH1B | Alcohol dehydrogenase 1B (class I), beta polypeptide |
| ADH4 | Alcohol dehydrogenase 4 (class II), pi polypeptide |
| AFM | Afamin |
| AGT | Angiotensinogen (serpin peptidase |
| | inhibitor, clade A, member 8) |
| AHSG | Alpha-2-HS-glycoprotein (Fetuin A) |
| AKR1C2 | Aldo-keto reductase family 1, member C2 |
| ALAS1 | Aminolevulinate, delta-, synthase 1 |
| ALDOB | Aldolase B, fructose-bisphosphate |
| AMBP | Alpha-1-microglobulin/bikunin precursor |
| ANGPTL4 | Angiopoietin-like 4 |
| AOX1 | Aldehyde oxidase 1 |
| APOA2 | Apolipoprotein A-II |
| APOB | Apolipoprotein B (including Ag(x) antigen) |
| APOC1 | Apolipoprotein C-I |
| APOE | Apolipoprotein E |
| APOH | Apolipoprotein H (beta-2-glycoprotein I) |
| APOM | Apolipoprotein M |
| AQP9 | Aquaporin 9 |
| ARG1 | Arginase, liver |
| ASGR2 | Asialoglycoprotein receptor 2 |
| ATF5 | Activating transcription factor 5 |
| BBC3 | BCL2 binding component 3 |
| BRP44L | Brain protein 44-like |
| C1S | Complement component 1, s subcomponent |
| C3 | Complement component 3 |
| C4A | Complement component 4A (Rodgers blood group) |
| C4BPA | Complement component 4 binding protein, alpha |
| C8A | Complement component 8, alpha polypeptide |
| CBR1 | Carbonyl reductase 1 |
| CFB | Complement factor B |
| CFH | Complement factor H |
| CFHR1 | Complement factor H-related 1 |
| CFHR2 | Complement factor H-related 2 |
| CFI | Complement factor I |
| CLU | Clusterin |
| CP | Ceruloplasmin (ferroxidase) |
| CPS1 | Carbamoyl-phosphate synthetase 1, mitochondrial |
| CRP | C-reactive protein, pentraxin-related |
| CYB5A | Cytochrome b5 type A (microsomal) |
| CYP2C9 | Cytochrome P450, family 2, subfamily C, polypeptide 9 |
| CYP2E1 | Cytochrome P450, family 2, subfamily E, polypeptide 1 |
| CYP3A5 | Cytochrome P450, family 3, subfamily A, polypeptide 5 |
| DDR2 | Discoidin domain receptor tyrosine kinase 2 |
| EPHA1 | EPH receptor A1 |
| ERRFI1 | ERBB receptor feedback inhibitor 1 |
| F11 | Coagulation factor XI (plasma thromboplastin |
| | antecedent) |
| F12 | Coagulation factor XII (Hageman factor) |
| F2 | Coagulation factor II (thrombin) |
| F9 | Coagulation factor IX |
| FGA | Fibrinogen alpha chain |
| FGB | Fibrinogen beta chain |
| FGG | Fibrinogen gamma chain |
| FGL1 | Fibrinogen-like 1 |
| FOS | V-fos FBJ murine osteosarcoma viral oncogene homolog |
| FTCD | Formiminotransferase cyclodeaminase |
| GABARAPL3 | GABA(A) receptors associated protein like 3 |
| GAPDH | Glyceraldehyde-3-phosphate dehydrogenase |
| GC | Group-specific component (vitamin D binding protein) |
| GSTA2 | Glutathione S-transferase A2 |
| HAGH | Hydroxyacylglutathione hydrolase |
| HAMP | Hepcidin antimicrobial peptide |
| HP | Haptoglobin |
| HPN | Hepsin (transmembrane protease, serine 1) |
| HPX | Hemopexin |
| HSD11B1 | Hydroxysteroid (11-beta) dehydrogenase 1 |
| ICAM1 | Intercellular adhesion molecule-1 |
| ID1 | Inhibitor of differentiation-1 |
| IL10 | Interleukin-10 |
| IL18 | Interleukin-18 |
| ITIH1 | Inter-alpha (globulin) inhibitor H1 |
| ITIH4 | Inter-alpha (globulin) inhibitor H4 |
| | (plasma Kallikrein-sensitive glycoprotein) |
| KCNK7 | Potassium channel, subfamily K, member 7 |
| KNG1 | Kininogen 1 |
| LECT2 | Leukocyte cell-derived chemotaxin 2 |
| LPA | Lipoprotein, Lp(a) |
| LRP1 | Low density lipoprotein receptor-related protein 1 |
| MAT1A | Methionine adenosyltransferase I, alpha |
| MRC1 | Mannose receptor, C type 1 |
| MST1 | Macrophage stimulating 1 (hepatocyte growth factor-like) |
| MT1A | Metallothionein 1A |
| MT1B | Metallothionein 1B |
| MT1E | Metallothionein 1E |
| NR1I3 | Nuclear receptor subfamily 1, group I, member 3 |
| ORM1 | Orosomucoid 1 |
| ORM2 | Orosomucoid 2 |
| PCK2 | Phosphoenolpyruvate carboxykinase 2 (mitochondrial) |
| PLG | Plasminogen |
| PON3 | Paraoxonase 3 |
| POR | P450 (cytochrome) oxidoreductase |
| PRDX4 | Peroxiredoxin 4 |
| PXMP2 | Peroxisomal membrane protein 2, 22 kDa |
| PYROXD1 | Pyridine nucleotide-disulphide oxidoreductase domain 1 |
| RBP4 | Retinol binding protein 4, plasma |
| SAA4 | Serum amyloid A4, constitutive |
| SEPP1 | Selenoprotein P, plasma, 1 |
| SERPINA1 | Serpin peptidase inhibitor, clade A, member 1 |
| SERPINA6 | Serpin peptidase inhibitor, clade A, member 6 |
| SERPINC1 | Serpin peptidase inhibitor, clade C (antithrombin), |
| | member 1 |
| SERPIND1 | Serpin peptidase inhibitor, clade D (heparin cofactor), |
| | member 1 |
| SLC13A2 | Solute carrier family 13, member 2 |
| SLC27A5 | Solute carrier family 27 (fatty acid transporter), |
| | member 5 |
| SPP2 | Secreted phosphoprotein 2, 24 kDa |
| TF | Transferrin |
| TGFB1 | Transforming growth factor, beta 1 |
| THNSL2 | Threonine synthase-like 2 (S. cerevisiae) |
| TM4SF4 | Transmembrane 4 L six family member 4 |
| TNFA | Tumor necrosis factor-alpha |
| TNFSF14 | Tumor necrosis factor (ligand) superfamily, member 14 |
| TSPAN9 | Tetraspanin 9 |
| TTR | Transthyretin (prealbumin, amyloidosis type I) |
| TXN | Thioredoxin |
| UGT1A6 | UDP glucuronosyltransferase 1 family, polypeptide A4 |
| UGT2B7 | UDP glucuronosyltransferase 2 family, polypeptide B7 |
| VEGFA | Vascular endothelial growth factor A |
| VTN | Vitronectin |
| ZGPAT | Zinc finger, CCCH-type with G patch domain |

Table 2 below lists 28 genes whose expression levels were more than two-fold-downregulated in tumor-unaffected hepatic tissue compared to the expression in metastatic tissue and peripheral blood mononuclear cells from stage IV patients with CRC having systemic metastasis disease. In bold are ten genes whose expression levels were downregulated in liver parenchymal and non-parenchymal sinusoidal cells given the conditioned medium from cultured CRC cells (HT-29 CRC cell line). This gene subset was also selected for further analysis.

TABLE 2

| | Downregulated Genes |
|---|---|
| ACTG1 | Actin, gamma 1 |
| ARPC2 | Actin related protein 2/3 complex, subunit 2, 34 kDa |
| BMP7 | Bone morphogenetic protein-7 |
| CALM1 | Calmodulin 1 (phosphorylase kinase, delta) |
| CAPG | Capping protein (actin filament), gelsolin-like |
| CEACAM1 | Carcinoembryonic antigen-related cell adhesion molecule 1 |
| | (biliary glycoprotein) |
| COL18A1 | Collagen, type xviii, alpha 1 |
| ERBB2IP | Erbb2 interacting protein |
| H2AFY | H2A histone family, member Y |
| H3F3B | H3 histone, family 3B (H3.3B) |
| HIST3H3 | Histone cluster 3, H3 |
| IFITM2 | Interferon induced transmembrane protein 2 (1-8D) |
| IGF1 | Insulin-like growth factor-1 |
| ING1 | Inhibitor of growth family, member 1 |
| NCL | Nucleolin |
| NGF | Nerve growth factor-beta |
| NOS2 | Nitric oxide synthase 2, inducible |
| PLP2 | Proteolipid protein 2 (colonic epithelium-enriched) |
| PPIA | Peptidylprolyl isomerase A (cyclophilin A) |
| RPIA | Ribose 5-phosphate isomerase A |
| | (ribose 5-phosphate epimerase) |
| RPL10 | Ribosomal protein L10 |
| RPS23 | Ribosomal protein S23 |
| RPS27 | Ribosomal protein S27 (metallopanstimulin 1) |
| RPS3A | Ribosomal protein S3A |
| SDC1 | Syndecam-1 |
| TMSB10 | Thymosin, beta 10 |
| TUBA4A | Tubulin, alpha 4a |
| YWHAB | Tyrosine 3-monooxygenase/tryptophan 5-monooxygenase |
| | activation protein, beta polypeptide |

Table 3 below shows liver prometastatic gene families (Inflammatory, Immune Regulation, Metabolic Bioprotection, and Fibrogenic Regeneration) of the thirty-one, two-fold upregulated and two-fold down-regulated genes of Tables 1 and 2 whose altered expression level in tumor-unaffected hepatic tissue is associated with liver metastasis growth in patients with CRC. The functional gene classification activity was performed manually by accessing the Gene Oncology and PubMed databases and is based on known biopathological functions assigned individually to studied genes. Below in Table 3 are listed and sorted by functional categories these 31 liver prometastatic genes.

TABLE 3
Liver Prometastatic Genes Sorted by Functional Categories

| Metabolic | | | Fibrogenic |
|---|---|---|---|
| Bioprotection | Immune | Inflammatory | Regenerative |
| Genes | Regulation Genes | Genes | Genes |
| TXN | KNG1 | ID-1 | RPS27 |
| PRDX4 | CEACAM-1 | IL-18 | RPS23 |
| NOS2 | BMP-7 | TNF-alpha | DDR2 |
| GAPDH | SDC-1 | VEGF-A | TGFB1 |
| MTE1 | COL18-A1 | EPHA-1 | VTN |
| HP | IL-10 | TNFSF14 | NGF |
| CRP | ICAM-1 | CYP2E1 | IGF-1 |
| ERBB21P | MRC1 | ADH1B | |

A first teaching of the present invention concerns detecting and/or identifying metastasis-associated genes in the tumor-unaffected hepatic tissue of Stage-IV cancer patients with metastatic CRC. As discussed in connection with FIG. 1, comparative transcriptome profiling using RNA from hepatic CRC metastases, tumor-unaffected hepatic tissue, and peripheral mononuclear blood cells uncovers approximately 122 genes specifically over-expressed and approximately 28 genes specifically under-expressed, each group being more than two-fold overexpressed or under-expressed in tumor-unaffected hepatic tissue from Stage-IV cancer patients with metastatic CRC. These genes are identified in Tables 1-2 above. Upregulated and downregulated gene sets were selected for further analysis. Transcriptome profiling was obtained from surgically removed liver specimens and archived biopsies of patient tissue. Table 3 shows a subset of these liver-associated genes (over-expressed and under-expressed genes) isolated according to their association with cancer-related cellular functions of inflammation, immune regulation, metabolic bioprotection and regeneration, i.e., functional categories. Further laboratory tests were performed on this subset of liver-associated genes to categorize them according to additional prometastatic criteria including (1) altered expression level in tumor-unaffected hepatic tissue associated with liver metastasis growth in patients with CRC, (2) altered expression in cultured liver parenchymal and non-parenchymal cells exposed to soluble factors from cultured CRC cells, and (3) altered expression associated with experimental hepatic colonization and growth of circulating CRC cells in animal models of CRC.

Table 4 below shows actual clinical data taken from forty-five patients (29 patients with CRC and 16 without CRC) that were included in the study on the expression pattern of liver prometastatic genes in hepatic biopsies from patients with and without CRC where TNM indicates tumor node metastasis stage.

TABLE 4

| | Patients with CRC | | | |
|---|---|---|---|---|
| Clinical Parameters | No. | % | Patients without CRC | |
| Gender | | | | |
| Female | 10 | 34 | 8 | 50 |
| Male | 19 | 66 | 8 | 50 |
| Average Age | 58 | — | 57 | — |
| Metabolic Syndrome | 14 | 48 | 7 | 43 |
| Cholelithiasis | 0 | 0 | 16 | 10 |
| | | | | 0 |
| TNM stage | | | | |
| I | 0 | 0 | 0 | 0 |
| II | 0 | 0 | 0 | 0 |
| III | 12 | 42 | 0 | 0 |
| IV | 17 | 58 | 0 | 0 |
| Tumor localization | | | | |
| Left-sided Colon | 11 | 39 | 0 | 0 |
| Right-sided Colon | 10 | 34 | 0 | 0 |
| Rectum | 6 | 20 | 0 | 0 |
| Others (gastric, duodenum) | 2 | 7 | 0 | 0 |

Table 5 below shows measurement data indicative of the thirty-one two-fold plus upregulated and down-regulated liver prometastatic gene expression levels under investigation in patients with and without CRC. The data shown therein represents average normalized (Ct Ratio of studied gene/Ct of constitutive gene) Ct (cycle threshold) values SD (Standard Deviation) as well as mean probability values “p-values.”

TABLE 5

| | | | Average | [symbol] -Student [symbol] U-Mann [symbol] Whitney [symbol] | |
|---|---|---|---|---|---|
| | | | Ct [symbol] Norm. [symbol] | [symbol] ( [symbol] [symbol] Value) | [symbol] ( [symbol] [symbol] Value) [symbol] |
| | | Without [symbol] CR [symbol] [symbol] | 0.812 | | |
| [symbol] | TXN | With [symbol] CR [symbol] [symbol] | 0.856 | 0.549 | 0.436 |
| | | Without [symbol] CR [symbol] [symbol] | 0.852 | | |
| [symbol] | PRDX4 | With [symbol] CR [symbol] [symbol] | 0.878 | | 0.086 |
| | | Without [symbol] CR [symbol] [symbol] | 0.892 | | |
| [symbol] | MT1E | With [symbol] CR [symbol] [symbol] | 0.970 | 0.026 | 0.014 |
| | | Without [symbol] CR [symbol] [symbol] | 1.000 | | |
| [symbol] | ERBB2IP | With [symbol] CR [symbol] [symbol] | 0.965 | 0.010 | 0.010 |
| | | Without [symbol] CR [symbol] [symbol] | 0.949 | | |
| [symbol] | NOS2 | With [symbol] CR [symbol] [symbol] | 1.175 | | 0.084 |
| | | Without [symbol] CR [symbol] [symbol] | 1.193 | | |
| [symbol] | HP | With [symbol] CR [symbol] [symbol] | 0.624 | 0.775 | 0.335 |
| | | Without [symbol] CR [symbol] [symbol] | 0.627 | | |
| [symbol] | CRP | With [symbol] CR [symbol] [symbol] | 0.833 | 0.004 | 0.002 |
| | | Without [symbol] CR [symbol] [symbol] | 0.908 | | |
| [symbol] | BMP7 | With [symbol] CR [symbol] [symbol] | 1.327 | 0.514 | 0.348 |
| | | Without [symbol] CR [symbol] [symbol] | 1.309 | | |
| [symbol] | SDC1 | With [symbol] CR [symbol] [symbol] | 0.921 | | 0.045 |
| | | Without [symbol] CR [symbol] [symbol] | 0.893 | | |
| [symbol] | IGF1 | With [symbol] CR [symbol] [symbol] | 0.975 | 0.593 | 0.741 |
| | | Without [symbol] CR [symbol] [symbol] | 0.970 | | |
| [symbol] | COL18A1 | With [symbol] CR [symbol] [symbol] | 0.842 | | 0.013 |
| | | Without [symbol] CR [symbol] [symbol] | 0.821 | | |
| [symbol] | ICAM1 | With [symbol] CR [symbol] [symbol] | 0.965 | | 0.000 |
| | | Without [symbol] CR [symbol] [symbol] | 1.019 | | |
| [symbol] | KNG1 | With [symbol] CR [symbol] [symbol] | 0.794 | | 0.042 |
| | | Without [symbol] CR [symbol] [symbol] | 0.776 | | |
| [symbol] | IL10 | With [symbol] CR [symbol] [symbol] | 1.081 | 0.000 | 0.000 |
| | | Without [symbol] CR [symbol] [symbol] | 1.130 | | |
| [symbol] | CEACAM1 | With [symbol] CR [symbol] [symbol] | 0.963 | 0.878 | 0.864 |
| | | | 0.962 | | |
| [symbol] | MRC1 | Without [symbol] CR [symbol] [symbol] | 0.976 | | 0.000 |
| | | With [symbol] CR [symbol] [symbol] | 1.018 | | |
| [symbol] | EPHA1 | Without [symbol] CR [symbol] [symbol] | 1.032 | 0.002 | 0.002 |
| | | WithCR [symbol] | 1.002 | | |
| [symbol] | TNFSF14 | Without [symbol] CR [symbol] [symbol] | 0.988 | | 0.025 |
| | | With [symbol] CR [symbol] [symbol] | 1.018 | | |
| [symbol] | CYP2E1 | Without [symbol] CR [symbol] [symbol] | 0.717 | 0.000 | 0.000 |
| | | With [symbol] CR [symbol] [symbol] | 0.688 | | |
| [symbol] | ADH1B | Without [symbol] CR [symbol] [symbol] | 0.734 | 0.001 | 0.001 |
| | | With [symbol] CR [symbol] [symbol] | 0.698 | | |
| [symbol] | ID1 | Without [symbol] CR [symbol] [symbol] | 0.908 | 0.007 | 0.001 |
| | | With [symbol] CR [symbol] [symbol] | 0.935 | | |
| [symbol] | TNF | Without [symbol] CR [symbol] [symbol] | 1.060 | 0.000 | 0.000 |
| | | With [symbol] CR [symbol] [symbol] | 1.164 | | |
| [symbol] | IL18 | Without [symbol] CR [symbol] [symbol] | 1.052 | 0.000 | 0.000 |
| | | With [symbol] CR [symbol] [symbol] | 1.114 | | |
| [symbol] | VEGFA | Without [symbol] CR [symbol] [symbol] | 0.910 | 0.882 | 0.792 |
| | | With [symbol] CR [symbol] [symbol] | 0.911 | | |
| | RPL23 | Without [symbol] CR [symbol] [symbol] | 0.807 | 0.766 | 0.712 |
| | | With [symbol] CR [symbol] [symbol] | 0.808 | | |
| | RPS27 | Without [symbol] CR [symbol] [symbol] | 0.763 | 0.148 | 0.178 |
| | | With [symbol] CR [symbol] [symbol] | 0.755 | | |
| | VTN | Without [symbol] CR [symbol] [symbol] | 0.707 | | 0.428 |
| | | With [symbol] CR [symbol] [symbol] | 0.702 | | |
| | NGF | Without [symbol] CR [symbol] [symbol] | 1.141 | 0.019 | 0.007 |
| | | With [symbol] CR [symbol] [symbol] | 1.113 | | |
| | TGFB1 | Without [symbol] CR [symbol] [symbol] | 0.972 | 0.934 | 0.989 |
| | | With [symbol] CR [symbol] [symbol] | 0.973 | | |
| | DDR2 | Without [symbol] CR [symbol] [symbol] | 1.045 | 0.438 | 0.421 |
| | | | 1.038 | | |

[symbol]

indicates data missing or illegible when filed

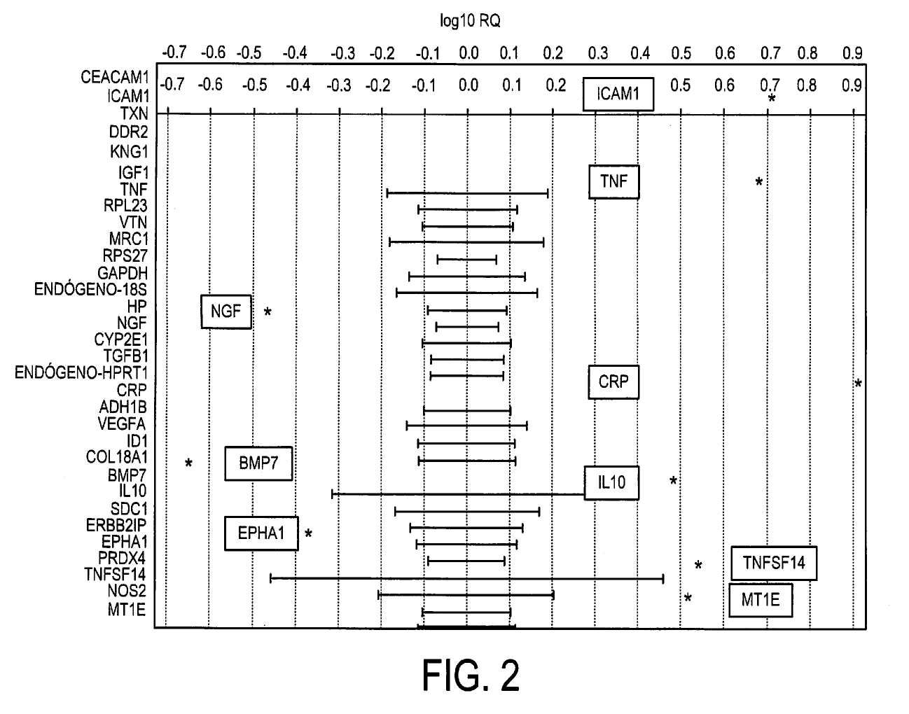

FIG. 2 depicts a logarithmic scale representation of the relative quantification (RQ) values of liver prometastatic gene expressions in CRC patients with respect to same values in patients without CRC. Error bars indicate maximum and minimum RQ values. “*” indicates statistically significant values where probability p<0.05 (i.e., less than five percent)

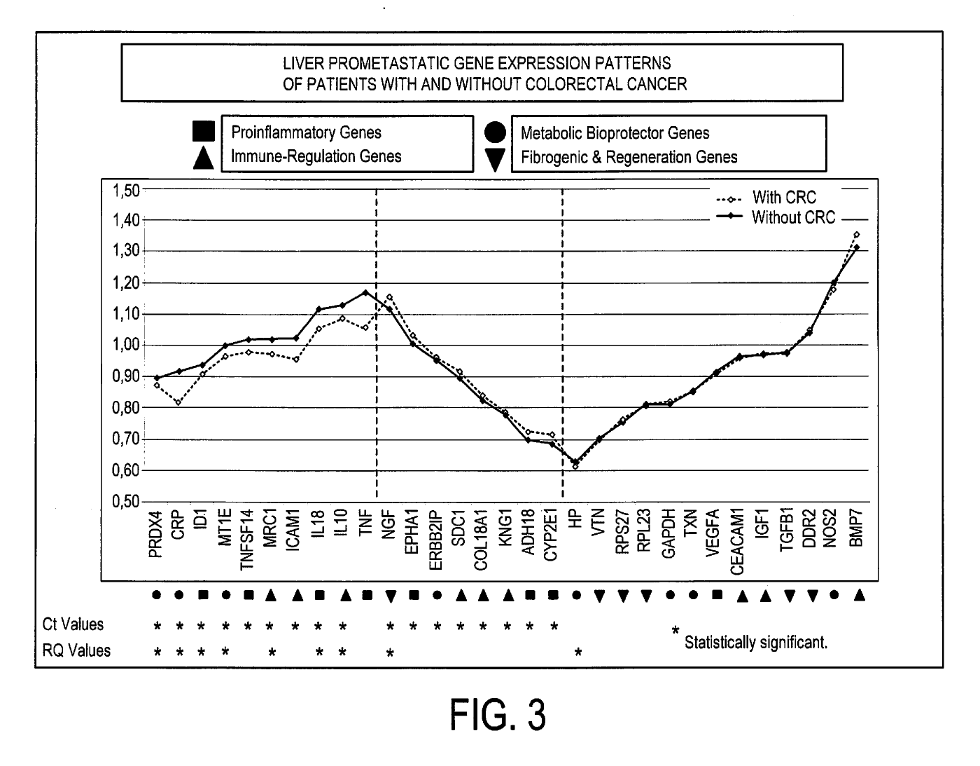

FIG. 3 depicts liver prometastatic gene expression patterns (color-coded in the related provisional applications according to functional category but here, ▪ indicates proinflammatory genes, ▴ indicates immune-regulation genes, ● indicates metabolic bioprotection genes, and ▾ indicates fibrogenic and regeneration genes) in patients with and without CRC. Differences between patients with and without CRC were statistically significant according to a U-Man Whitney test (p<0.05). Statistically significant genes are identified by “*” denoted in the lower legend of FIG. 3. Advantageously, by examining the expression levels of one or more statistically significant genes in group 1 and/or group 2 of patients with CRC relative to a control (i.e., gene expression levels of persons free of CRC), rather than waiting for clinical signs to become apparent by imaging or non-biochemical changes in the microenvironment, one may detect metastatic cancer in the hepatic biochemical microenvironment to enable very early treatment and potential eradication of metastatic cancer cells.

An aspect of the invention includes a complementary diagnostic test to detect “liver prometastatic reaction level and class” in patients with CRC without metastatic disease. Expression of liver prometastatic genes in hepatic tissue selected above was next studied in twenty-nine patients with CRC (at stages III and IV) and sixteen patients without CRC used as controls. Table 4 details clinical information about the patients involved in the study. Based on normalized Ct values, Table 5 shows average values of the gene expression levels for the 31 genes involved for the 29 patients with CRC. As reflected in FIG. 3, expression levels were significantly (probability p<0.05) increased for ten liver prometastatic genes (group 1) and decreased in eight genes (group 2), with non-statistically significant (i.e., insignificant) changes in twelve to thirteen genes, when comparing patients with (29 patients) and without (16 patients) CRC. The vertical axis of FIG. 3 reflects the relative Cₜ data relative to a control, or ratio of the Cₜ cycle count (i.e., Ct value) of the sample under examination relative to the Cₜ cycle count of a control Cₜ value. Regarding group 1 genes of patients with CRC, i.e., the dotted trace, detection of an expression level signal at a lower cycle count Cₜ in the PCR process indicates a higher gene expression level. Detection of an expression level signal for each of the group 2 genes of patients with CRC occurred at a higher cycle count Cₜ as reflected in the middle portion of the dotted trace of FIG. 3. However, no statistically significant changes in gene expression levels of this latter group of twelve to thirteen genes (group 3) were detected when comparing CRC patients with and without hepatic metastases (or between CRC patients and CRC-free patients as controls) suggesting that detected liver prometastatic gene expression changes in tumor unaffected hepatic tissue nevertheless occurred in the liver of CRC patients irrespective of having or not having metastases. In addition, correlation of respective gene expression levels of group 3 genes was deemed required in order to validate the efficacy of the relative expression levels of group 1 and group 2 genes. In other words, without congruence of the respective expression levels of patients with and without CRC among group 3 genes, the conditions denoted for group 1 and group 2 genes would not be valid.

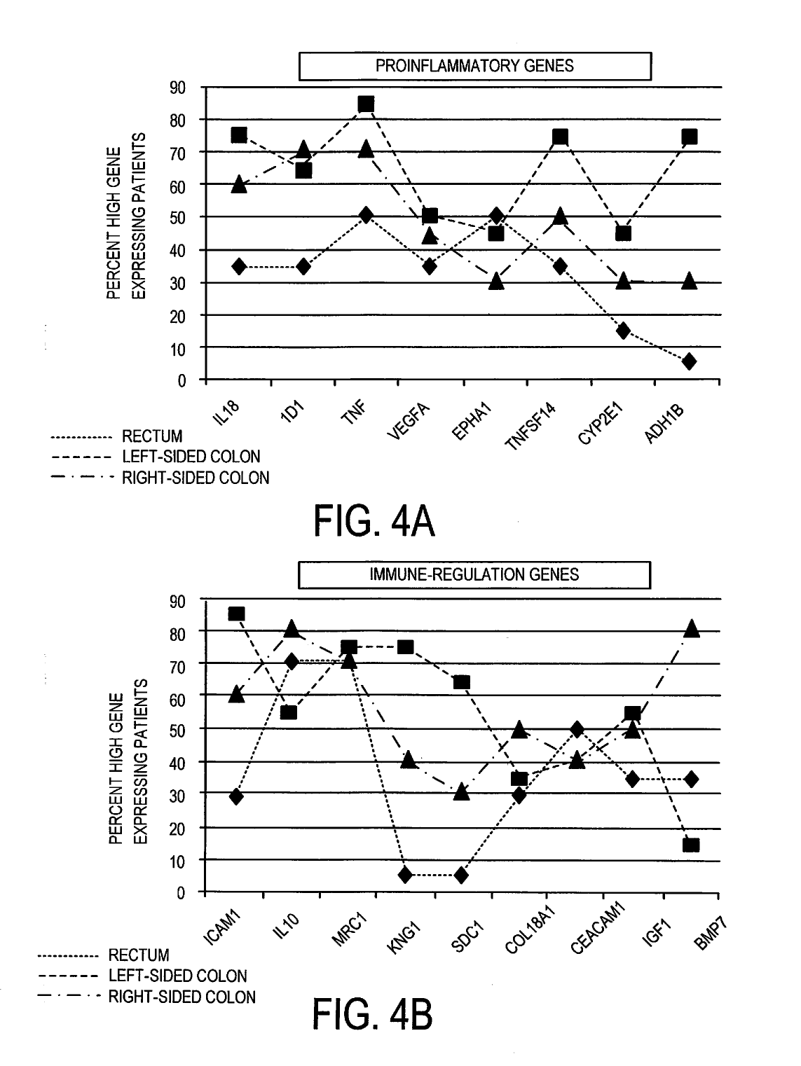

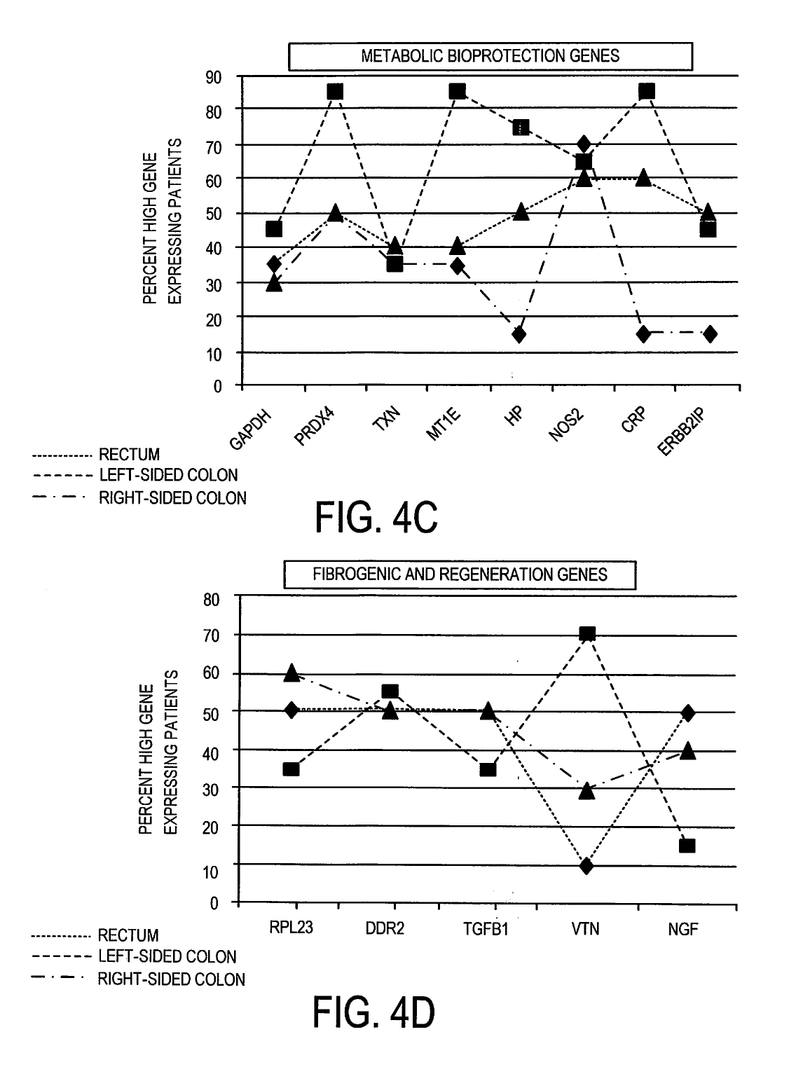

FIGS. 4A-4D and Table 6 further indicate that expression levels of the various genes differ according to the anatomical location of the patient's primary CRC, i.e., in the rectum, left-side colon or the right-side colon. Therefore, a liver prometastatic reaction occurs in the liver of patients with CRC prior to metastasis development and, in accordance with another aspect of the invention, by scoring number and intensity of gene changes according to the relationships shown in FIGS. 4A-4D, we may determine the genesis of the prometastatic hepatic cancer risk.

In addition, a majority of proinflammatory (seven out of eight) and immune regulation (six out of nine) liver prometastatic genes, but only a minority of fibro-regenerative (one out of five) and metabolic bio-protective (three out genes eight) were significantly (p<0.05) changed in patients with CRC versus patients without CRC (Table 3, Table 6, and FIGS. 4A-4D). This suggests that in addition to the number of changed genes, the kind of changed genes in functional terms defines the Liver Prometastatic Reaction Class in the liver of patients with CRC. Both number and functional categories of liver prometastatic genes changed in patients with CRC may serve as a complementary diagnostic test for the quantitative assessment of liver metastasis risk and recurrence in patients with CRC, and thus, as a precursor, may form the basis of a method of detecting occult CRC subclinically in patients having no clinical symptoms of CRC at all. A processing system to receive data inputs and appropriate program instructions may be utilized to automatically output this determination on a display device or other output.

TABLE 6
Liver prometastatic gene expression level by functional
category and anatomic location of the primary CR

[symbol]

| | Gene | | LEFT- | RIGHT- |
|---|---|---|---|---|
| Liver Prometastatic | Expression | | SIDED | SIDED |
| Genes | Level | RECTUM | COLON | COLON |
| PRO- | HIGH | — | IL18 | ID1 |
| INFLAMMATORY | | | ID1 | TNF |
| GENES | | | TNF | |
| | | | TNFSF14 | |
| | | | ADH1B | |
| | LOW | IL18 | — | ADH1B |
| | | ID1 | | CYP2E1 |
| | | VEGFA | | |
| | | TNFSF14 | | |
| | | ADH1B | | |
| | | CYP2E1 | | |
| IMMUNO | HIGH | IL10 | ICAM1 | IL10 |
| REGULATION | | MRC1 | MRC1 | MRC1 |
| GENES | | | KNG1 | BMP7 |
| | | | SDC1 | |
| | LOW | ICAM1 | BMP7 | KNG1 |
| | | KNG1 | | SOC1 |
| | | SOC1 | | |
| | | IGF1 | | |
| | | BMPT | | |
| METABOLIC | HIGH | NOS2 | PRXD4 | — |
| BIOPROTECTION | | | MTE1 | |
| GENES | | | HP | |
| | | | NOS2 | |
| | | | CRP | |
| | LOW | GAPDH | TXN | — |
| | | TXN | | |
| | | MTE1 | | |
| | | HP | | |
| | | CRP | | |
| | | ERSB2IP | | |
| PEROGENIC AND | HIGH | — | VTN | — |
| REGENERATION | | | | |
| GENES | | | | |
| | LOW | VTN | RLP23 | VTN |
| | | | TGFB1 | NGF |
| | | | NGF | |

[symbol]

indicates data missing or illegible when filed

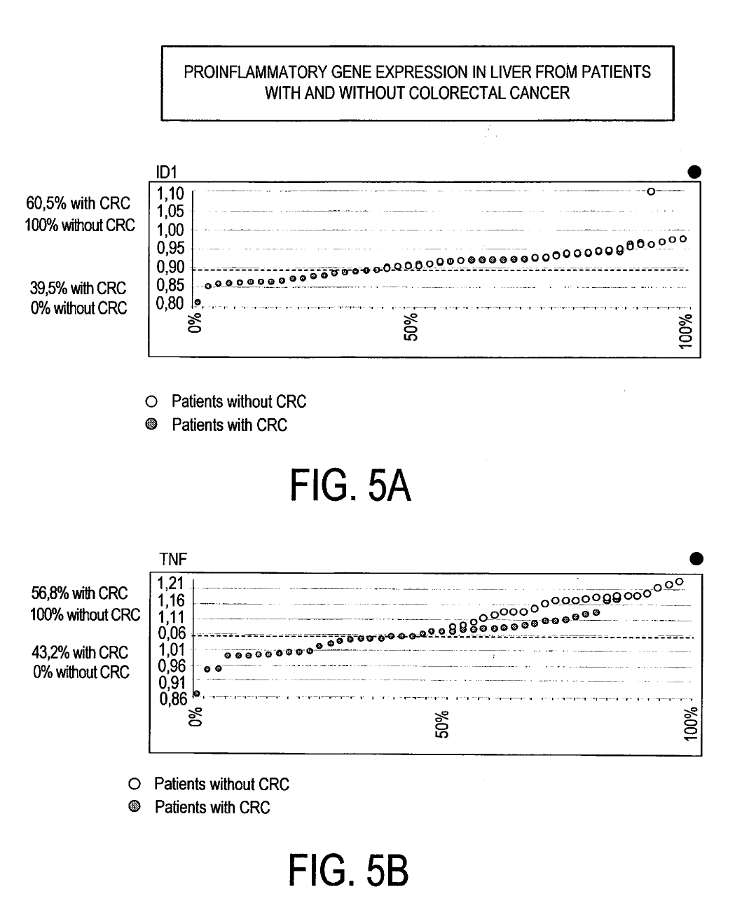

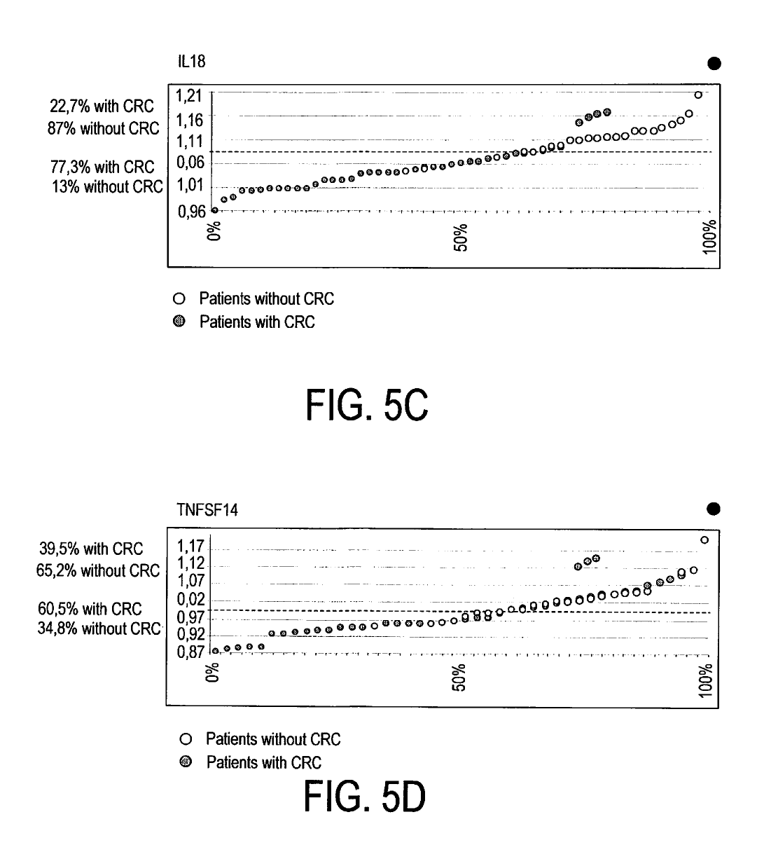

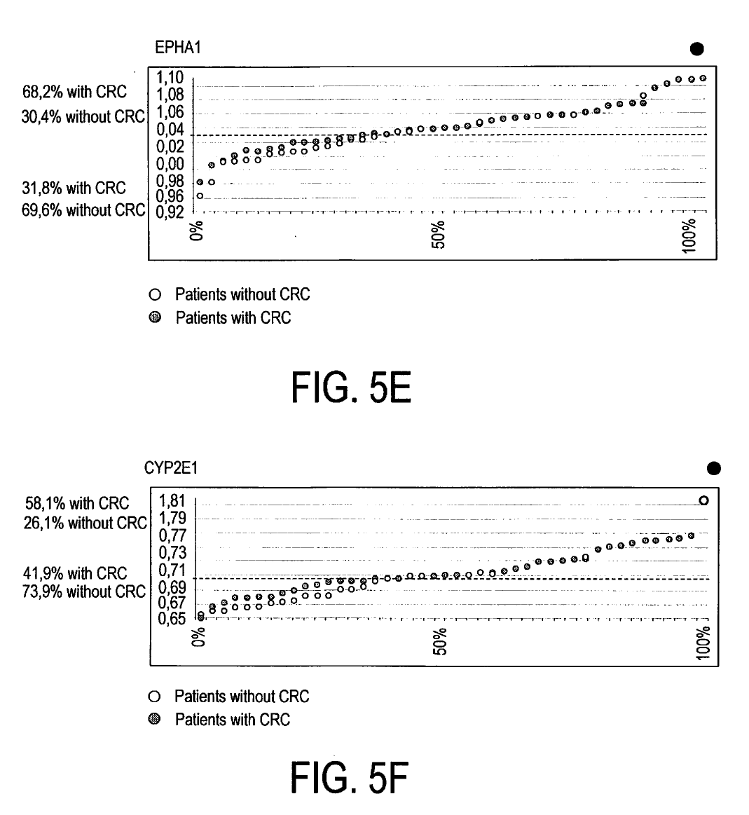

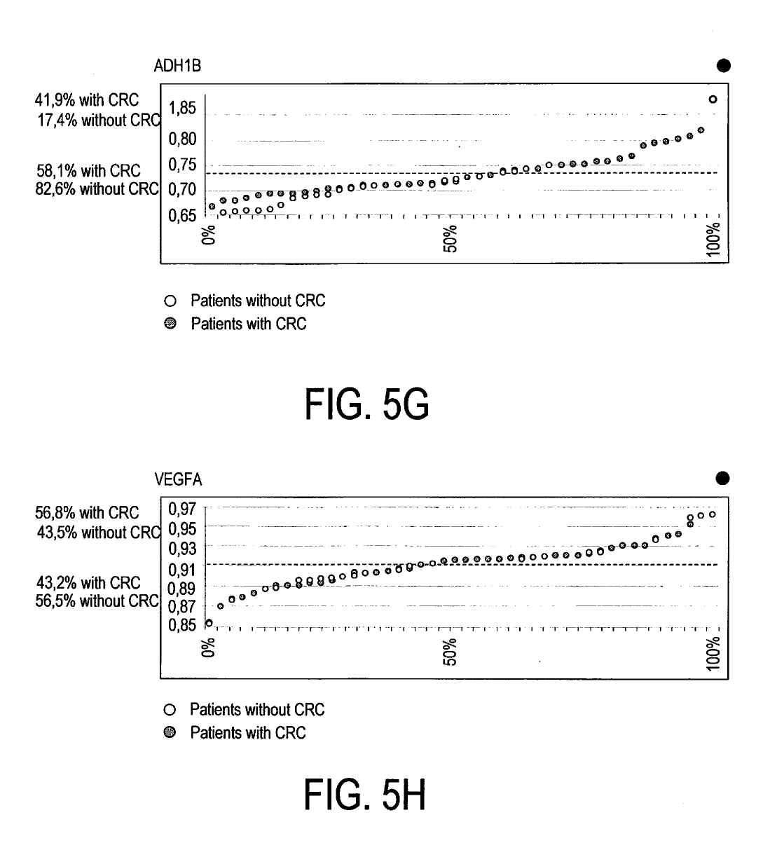

FIGS. 5A-5H show proinflammatory gene expressions of respective genes denoted in the charts in liver from patients with and without CRC and how, if at all, they differ among the various patients. Data are represented as increasing distribution of mean values. Data express normalized Ct values (ration of Ct of studied gene/Ct of constitutive gene). The discontinuous line marks the intermediate point between the minimum and maximum ratios obtained for each gene (Y axis) for total number of analyzed samples (X axis) from patients with and without CRC.

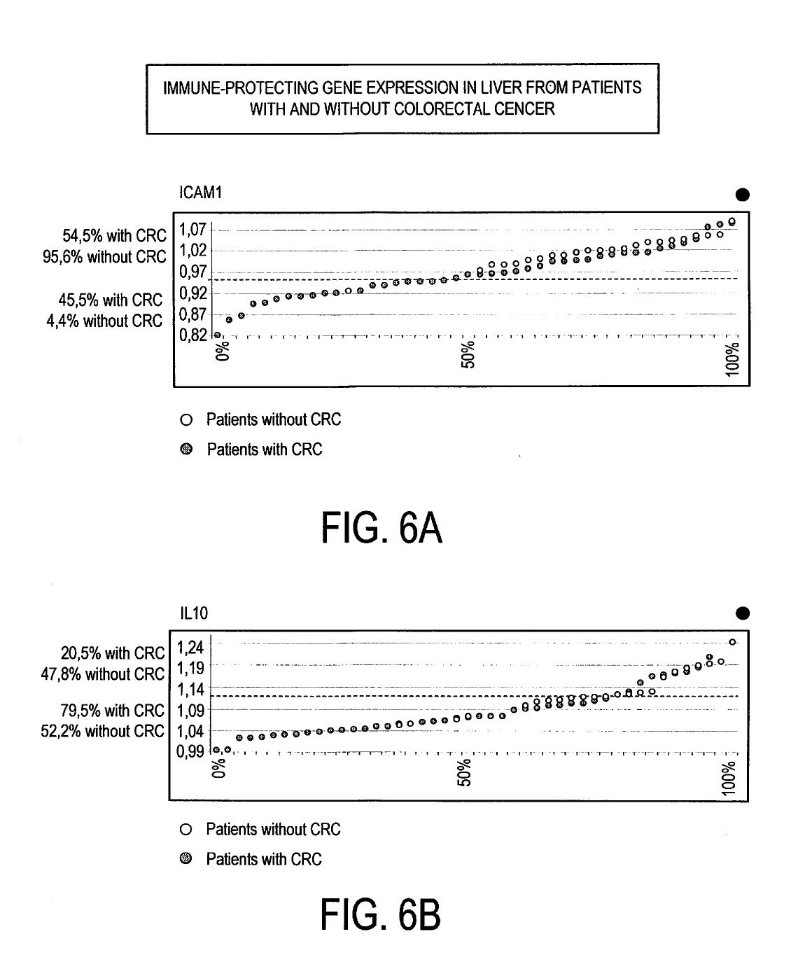

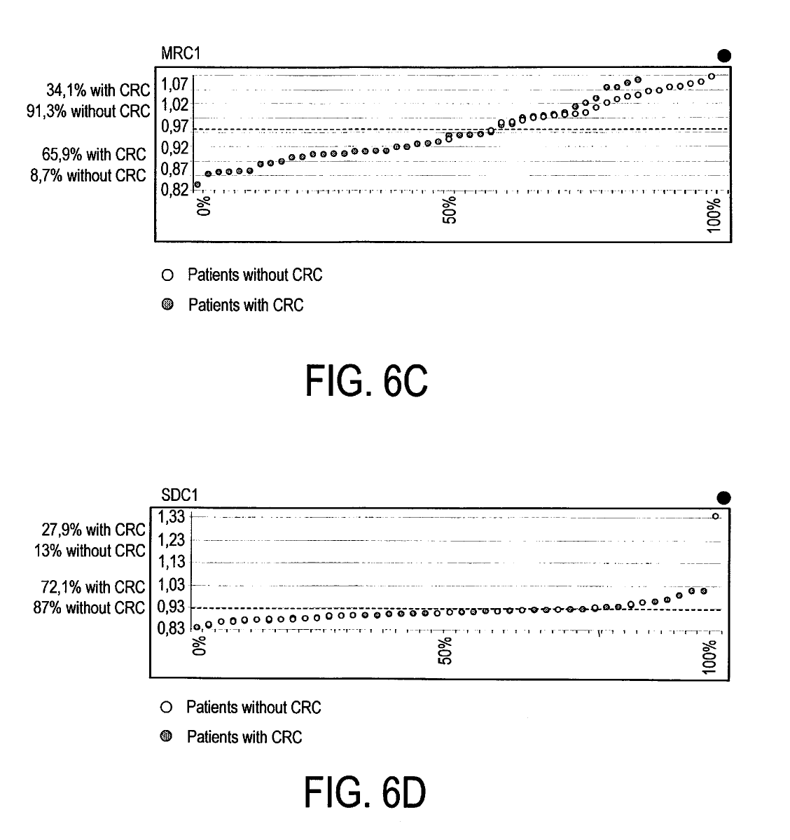

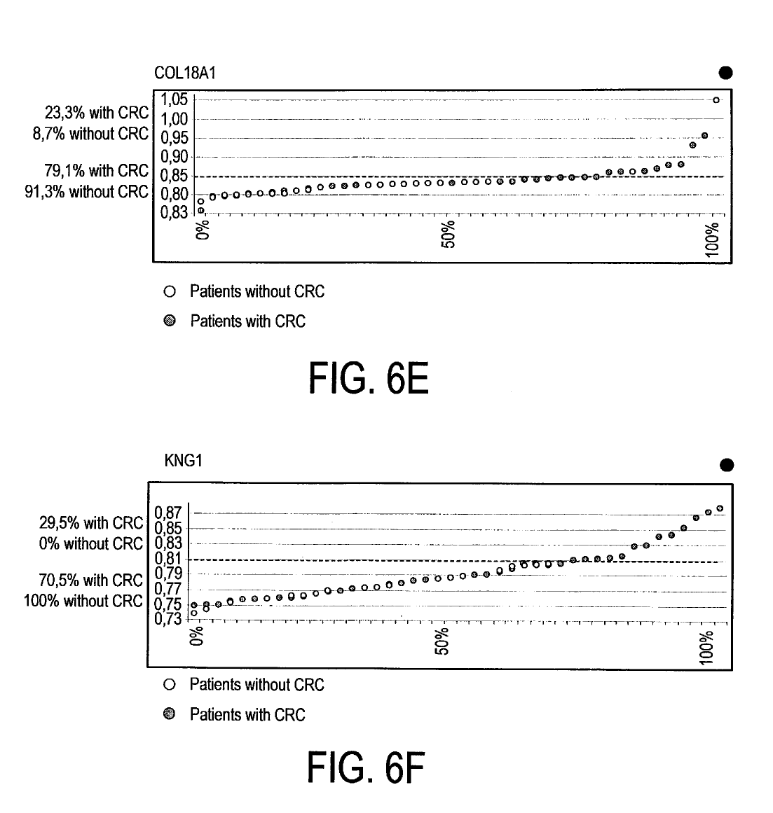

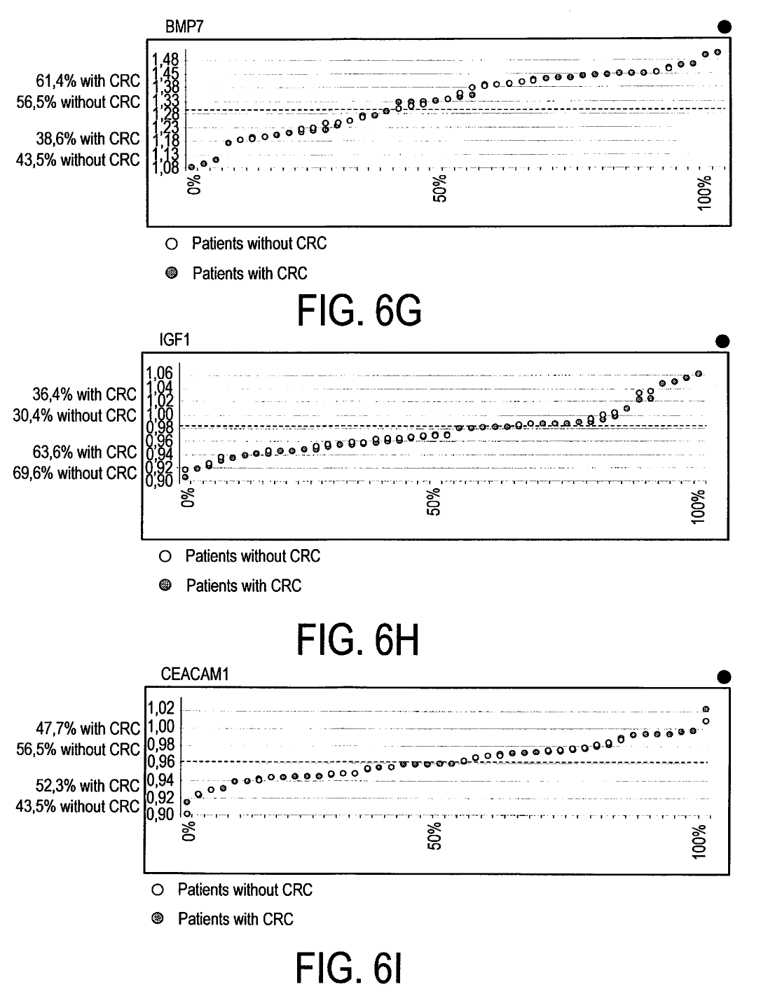

FIGS. 6A-6I similarly show immuno-regulation gene expressions of respective genes denoted in the charts in liver from patients with and without CRC, as well as how if all they differ. Data are represented as increasing distribution of mean values. Data express normalized Ct values (ration of Ct of studied gene/Ct of constitutive gene). The discontinuous line marks the intermediate point between the minimum and maximum ratios obtained for each gene (Y axis) for total number of analyzed samples (X axis) from patients with and without CRC.

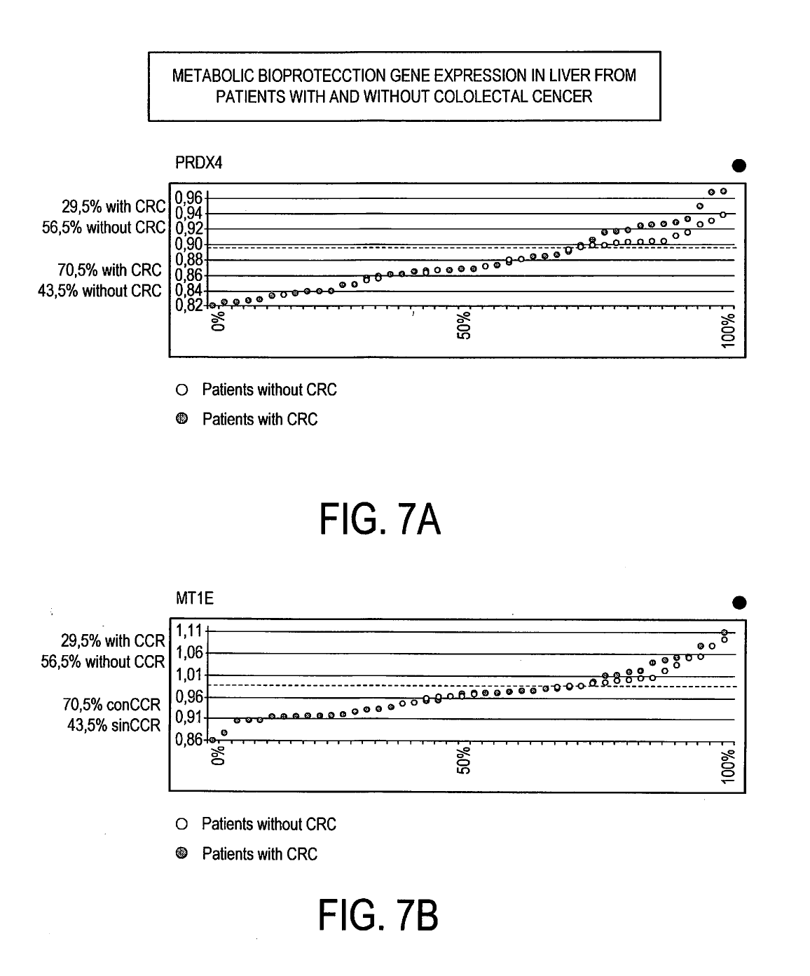

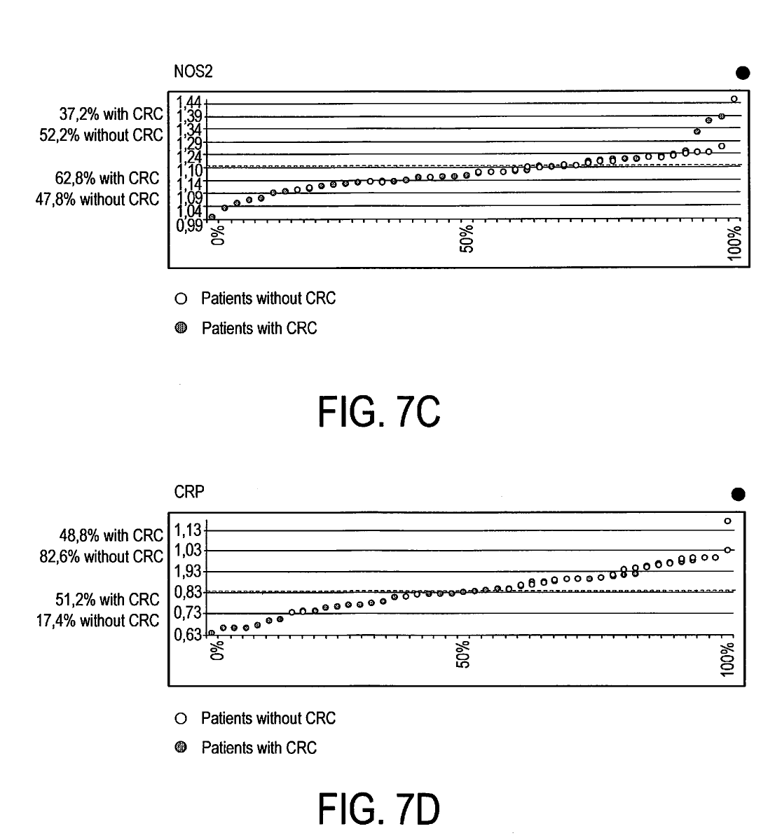

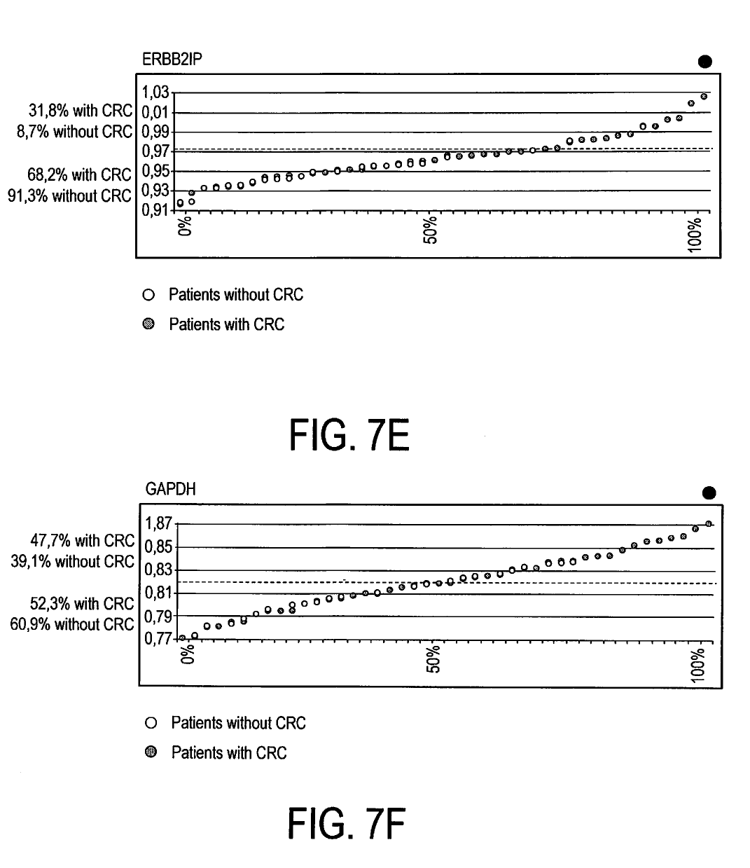

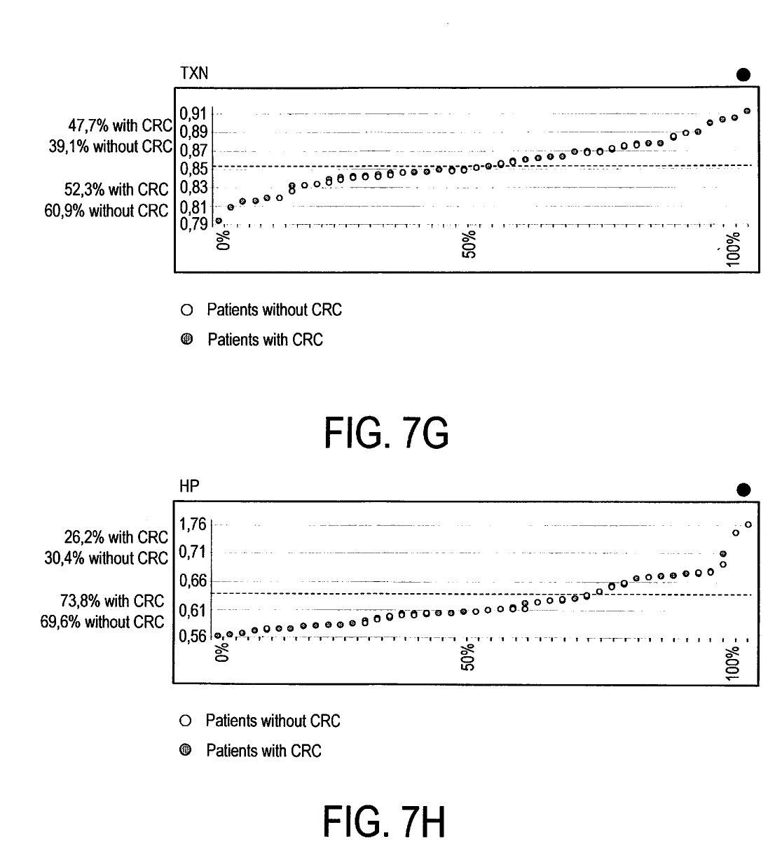

FIGS. 7A-7H similarly show metabolic bioprotection gene expressions of denoted genes in liver from patients with and without CRC, as well as, how they differ among patients. Data are represented as increasing distribution of mean values. Data express normalized Ct values (ratio of Ct of studied gene/Ct of constitutive gene). The discontinuous line marks the intermediate point between the minimum and maximum ratios obtained for each gene (Y axis) for total number of analyzed samples (X axis) from patients with and without CRC.

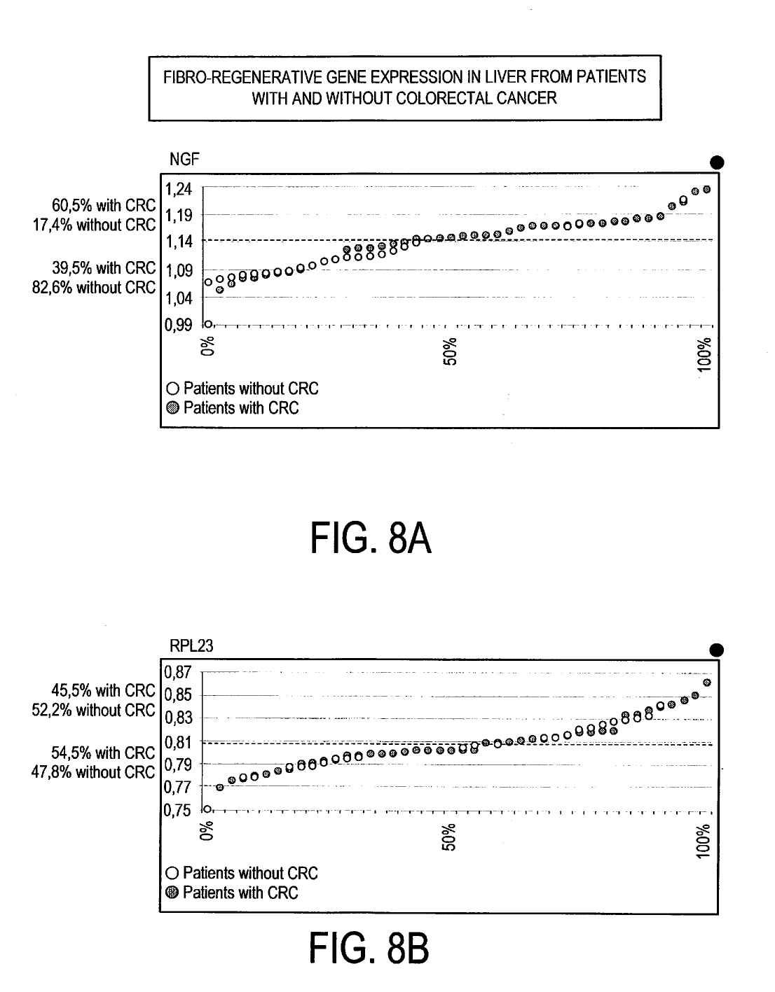

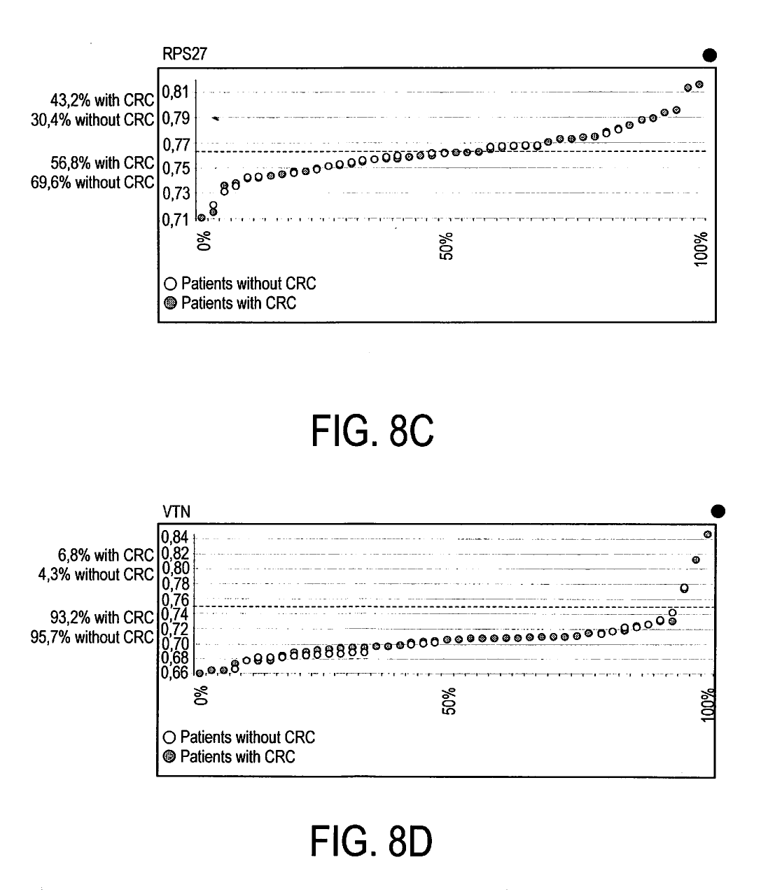

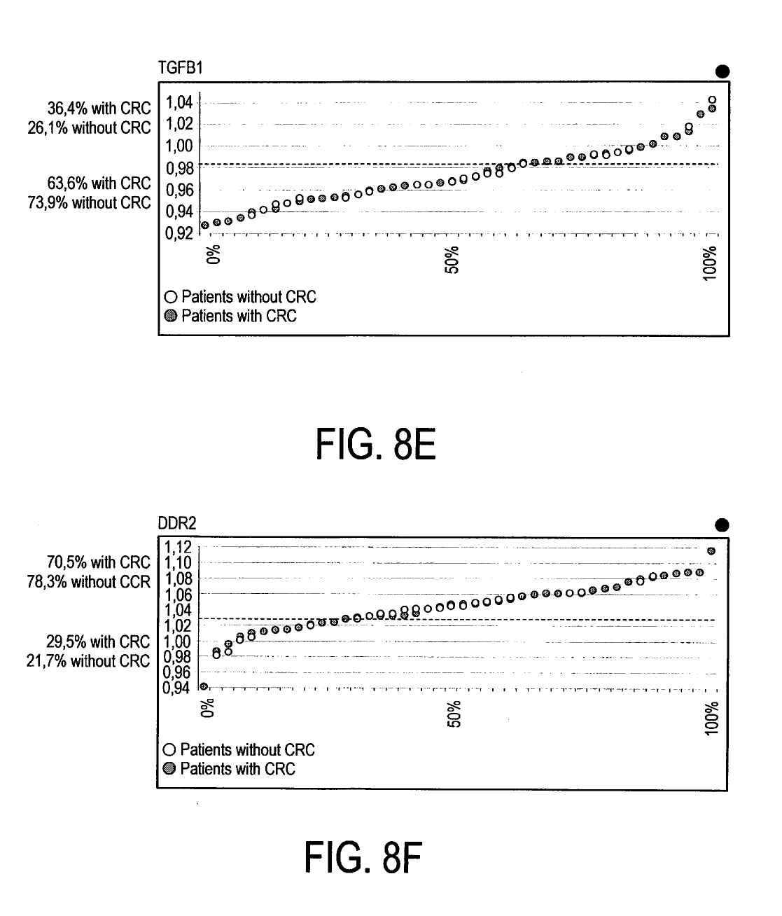

FIG. 8A-8F similarly show depicts fibrogenic and regeneration gene expressions of denoted genes in liver from patients with and without CRC, as well as, how they differ among patients, Data are represented as increasing distribution of mean values. Data express normalized Ct values (ratio of Ct of studied gene/Ct of constitutive gene). The discontinuous line marks the intermediate point between the minimum and maximum ratios obtained for each gene (Y axis) for total number of analyzed samples (X axis) from patients with and without CRC.

Based on analyses illustrated in FIGS. 5-8, another aspect of the invention includes a complementary diagnostic test to provide an alert of possible occult CRC in patients without clinical evidence of CRC but with other digestive system diseases that increase CRC risk, such as, but not limited to, cholelithiasis and metabolic syndrome. Comparative distribution of gene expression levels of selected genes of studied CRC patients and their controls without CRC, by their expression of liver prometastatic genes (as indicated by the analyses shown in FIGS. 5-8) demonstrate that those genes best contributing to the segregation of patients with and without CRC are Metabolic bioprotection genes PRDX4, MT1E, CRP and NOS2; Immune regulation genes ICAM1, IL10 and MRC1; and Proinflammatory genes ID1, TNF-a, IL18 and TNFSF14. All of these genes remarkably increased their expression levels in patients with CRC while decreased their expression levels in patients without CRC. On the contrary, immune-regulation genes SDC1, COL18A1 and KNG1, Proinflammatory genes EPHA1, CYP2E1, ADH1B, and fibrogenic/regeneration gene NGF increased their expression levels in patients without CRC while decreased their expression levels in patients with CRC, as indicated in FIGS. 5-8.

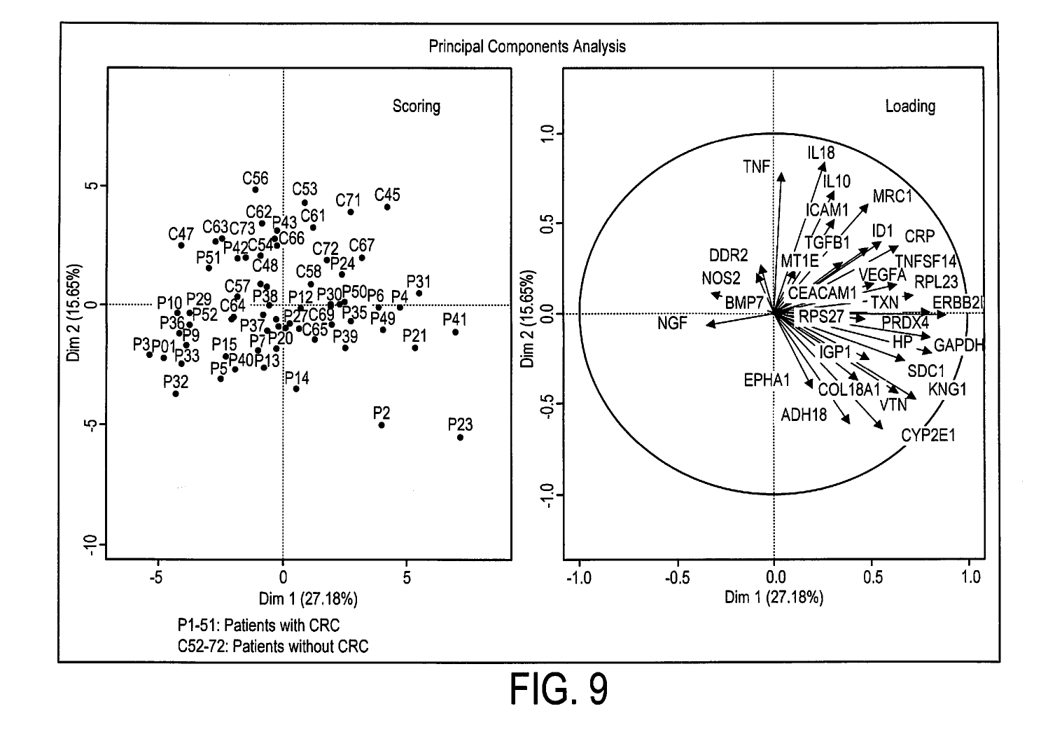

FIG. 9 shows a comparison of overall expression profiles across samples from patients with and without CRC in respective Scoring Value and Loading Value charts. Scoring chart of FIG. 9 shows results for a Principal Component Analyses (PCA) of the data, which is used to emphasize variations and reveal data patterns of gene expressions in patients (P1-P51) with and patients (C52-C72) without CRC. The first principal component (Dimension 1) sets forth a 27.18% variation, whereas the second Dimension 2 sets forth a 15.65% variation. It is seen that Dimension 2 separated most of patients with CRC from patients without CRC.

A principal component analysis (PCA), multivariate regression analysis used to distinguish samples with multiple measurements was conducted, the results of which are shown in FIG. 9. Supervised discriminant analysis showed that liver prometastatic Immune regulation and proinflammatory genes were the most discriminative for patients with and without CRC.

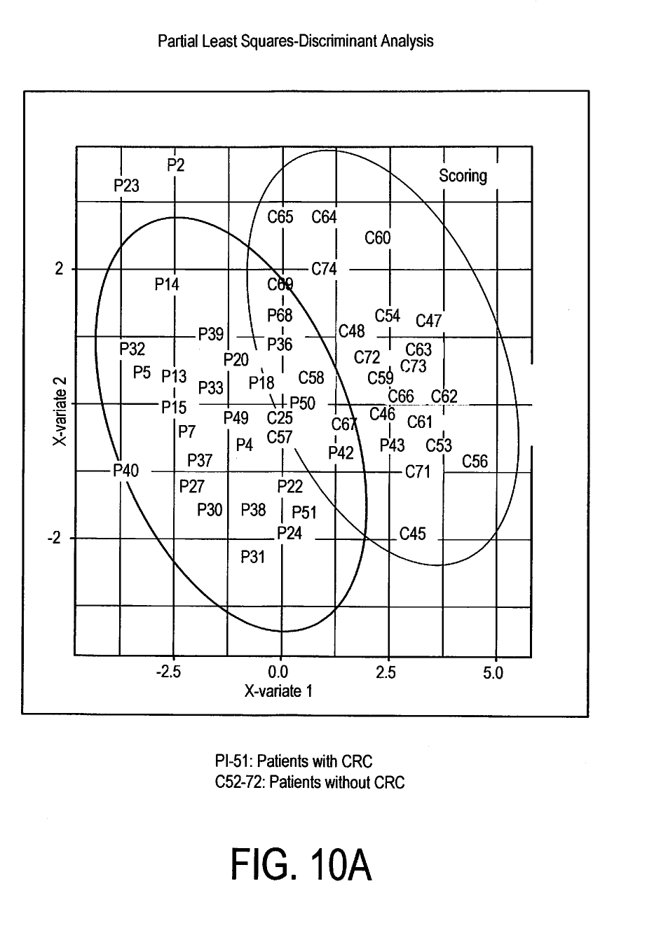

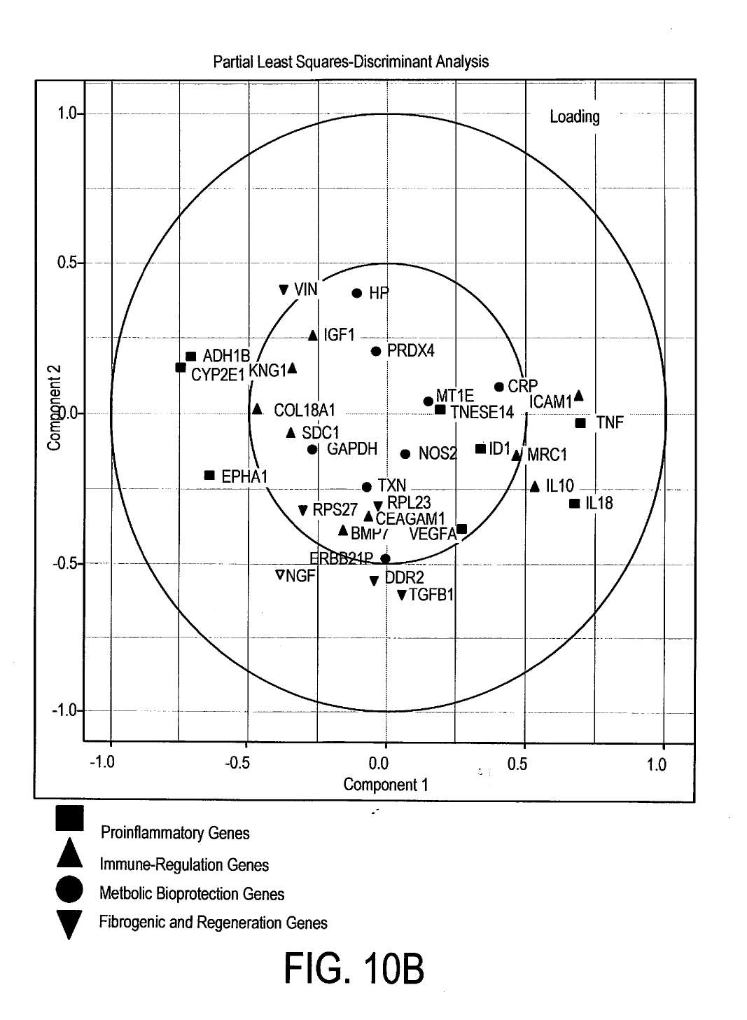

FIGS. 10A and 10B show a Partial Least Squares-Discriminant Analysis (PLS-DA) of gene expression data in respective Scoring and Loading plots, which are intended to discriminate between patients with and without CRC based on their hepatic expression levels of liver prometastatic genes. FIG. 10A depicts elliptical shapes adopted by lines that define the position coordinates of included patients (patients C52-72 without CRC and patients P1-51 with CRC). In this case, the discriminant capacity was associated with the first component in the analysis. FIG. 10B depicts position coordinates of liver prometastatic genes plotted in correlation circles whose diameters define influence of the genes in the prediction of the class of patient. In this case, metabolic bioprotection and fibrogenic/regeneration genes are in the smaller circle, indicating that their expression levels had less ability to predict the patient's class than Immune regulation and proinflammatory genes mainly located in the large correlation circle, which indicates a greater predictive capacity of the patient class.

FIGS. 10A and 10B show results of a supervised discriminant analysis to classify genes and patients by their correlation and ability to predict patients with and without CRC. The elliptical shapes adopted by lines in FIG. 10A define position coordinates of included patients and show that the discriminant capacity was associated with the first component in the analysis. Next, position coordinates of studied liver prometastatic genes were plotted in correlation circles (FIG. 10B), whose diameters define the influence of the genes in the prediction of the class of patient. Studied genes were distributed in correlation circles according to their functional category and once again, metabolic bioprotection and fibrogenic/regeneration genes were in the smaller circle, indicating that their expression levels had less ability to predict the patient's class, while Immune regulation and proinflammatory genes were mainly located in the large correlation circle, indicating their greater predictive capacity of the patient class.

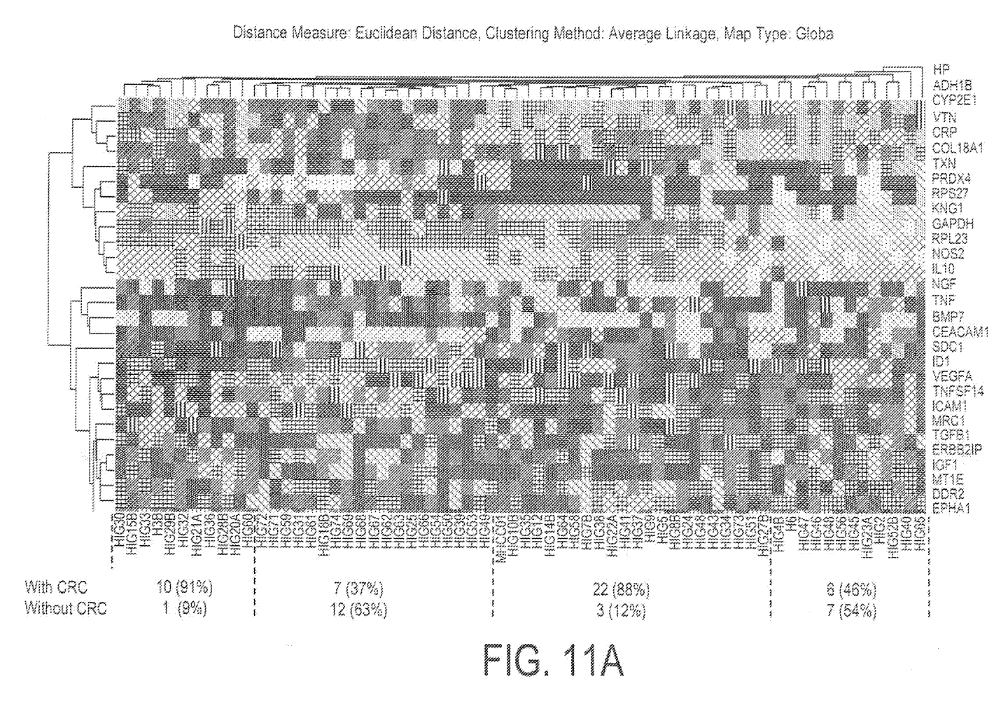

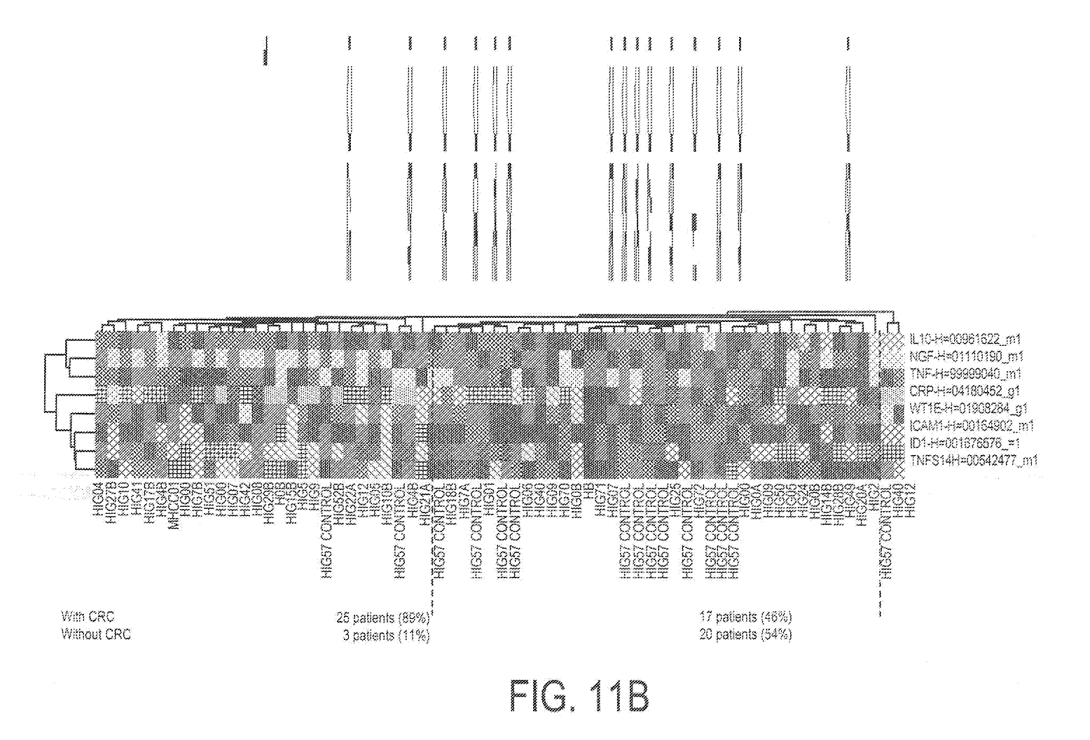

FIGS. 11A and 11B show heatmaps for clustering patients with and without CRC according their liver prometastatic gene expression patterns based on AACt ratio. FIG. 11A shows four subgroups of patients with distinct gene expression patterns, two of them being enriched by patients with CRC and two others by patients without CRC. FIG. 10B shows two subgroups with distinct gene expression patterns (genes with significant averages difference and significant RQ (relative quantization)), enriched by either patients with or without CRC that were generated using the most discriminating genes. Some patients (noted in color green in the first and third groups) without CRC are seen to be grouped with patients with CRC suggesting they may have occult CRC (which was later confirmed by colonoscopy), while other patients with CRC (as noted in the second and fourth groups) were grouped with patients without CRC. Interesting, none of these ectopic CRC patients had hepatic metastases. Thus, according to yet another aspect of the present invention, manifestations of clustering provide a basis for early subclinical detection and pretreatment of occult CRC in patients lacking clinical symptoms.

An unsupervised hierarchical cluster analysis was performed to determine whether aggregation of genes by their expression similarity level per patient contributed to segregation of patients with and without CRC. Application of Euclidean distances between studied genes resulted in the appearance of clusters allowing the distribution of patients according to their transcriptional similarity levels. As shown in FIG. 11A, the heatmap outlined four mixed subgroups of patients with distinct gene expression patterns, two of them enriched by patients with CRC and two others by patients without CRC. A new heatmap (FIG. 11B) was constructed using genes with the highest predictive power of the class of patient, as evidenced in the PLS-DA analysis. In this case, the power of discrimination was comparable to that obtained in the previous heatmap, but in this case there was a segregation in two large mixed subgroups rather than four, both of which being enriched either in patients with CRC or without CRC. Some patients without CRC are grouped with patients with CRC suggesting they may have occult CRC (which was later confirmed by colonoscopy), while some patients with CRC are grouped with patients without CRC (none of CRC patients had hepatic metastases).

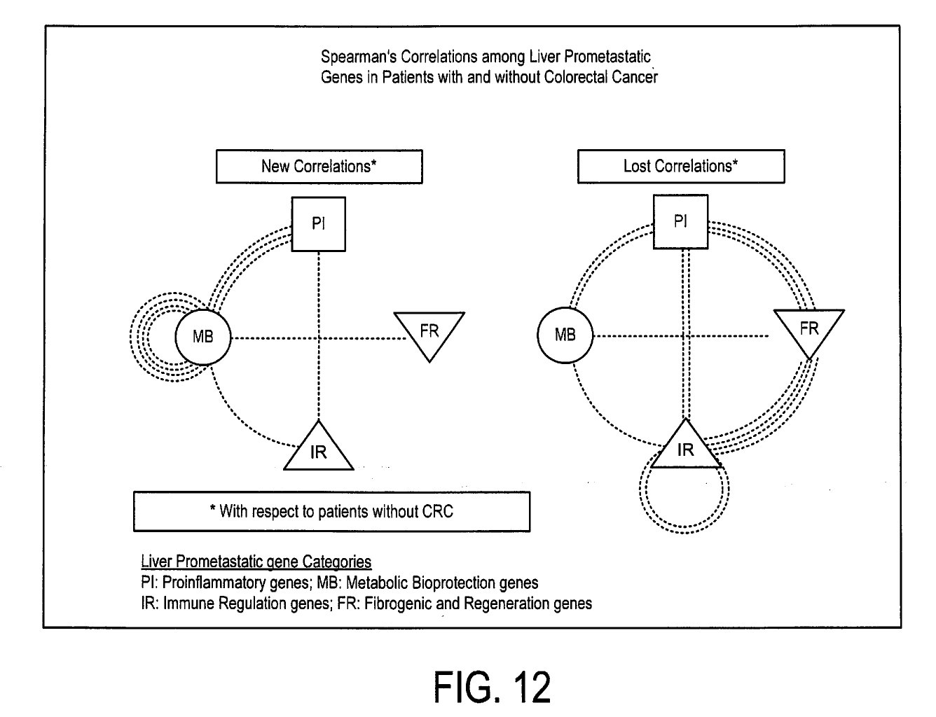

FIGS. 12A and 12B show Spearman's correlation of expression levels among liver prometastatic genes in patients with and without CRC. Only statistically significant (p<0.05 or higher) correlations with coefficient Rho equal to or greater than 0.7 were considered in this analysis. Nine of the ten correlations in patients with CRC involved five bioprotective genes (HP, ERBB2IP, GAPDH, CRP, PDRX4); four of these correlation gains were produced among bioprotective genes (ERBB2IP-GAPDH; ERBB2IP-PDRX4; CRP-GAPDH; CRP-HP), three among metabolic bioprotection and proinflammatory genes (CRP-TNFSF14; HP-TNFSF14; GAPDH-ID1) (CRP-NGF), immune-regulation genes (PDRX4-CEACAM1), as well as between proinflammatory and immune-regulation genes (TNFSF14-COL18A1). In contrast, eight out of the fourteen lost gene correlations in patients with CRC occurred in immune-regulation gene group (involving CEACAM1, MRC1, ICAM1, IL10, BMP7 genes), of which four were lost between immune-regulation and fibrogenic/regeneration genes (ICAM1-TGFB1; IL-10-NGF, CEACAM1-NGF, MRC1-NGF), whereas only two were lost between immune-regulation and proinflammatory genes (BMP7-TNF, CEACAM1-TNF) and another two among immune-regulation genes (CEACAM1-BMP7; MRC1-BMP7). There was also a striking loss of seven correlations between proinflammatory genes and other functional categories of genes (metabolic bioprotection, fibrogenic/regeneration and immune-regulation genes).

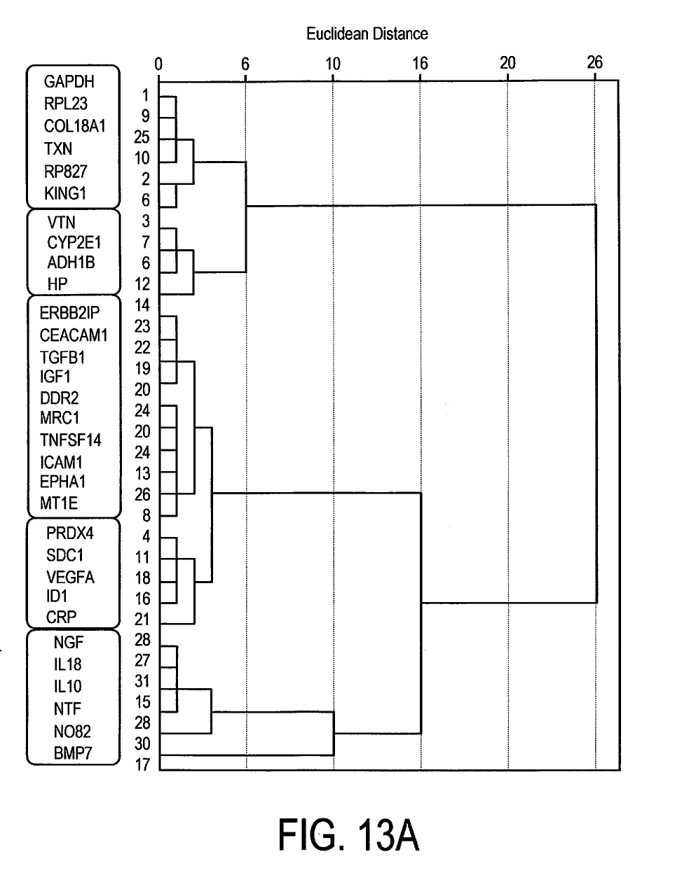

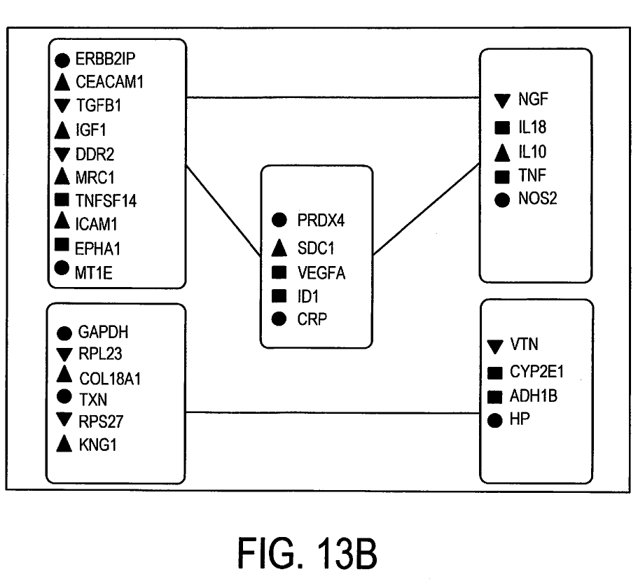

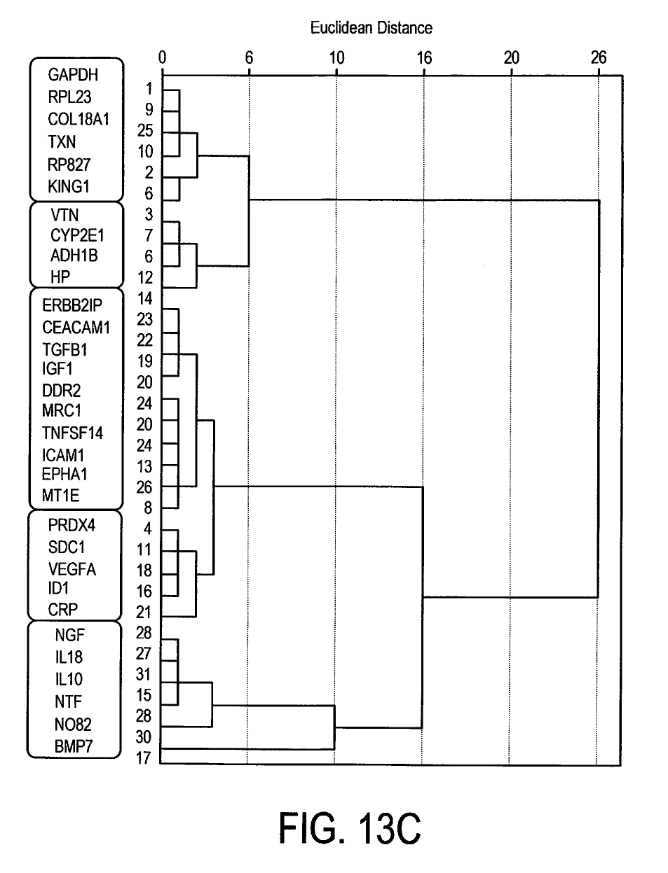

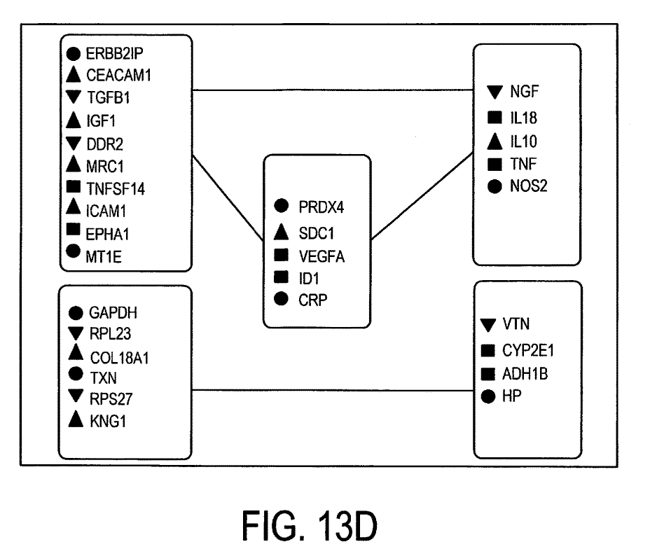

FIGS. 13A-13D show hierarchical clustering performed based on Pearson's correlation Euclidean distance among the genes and gene clusters, and the results presented as a dendrogram plot in order to define the transcriptional structure of prometastatic genes in hepatic biopsies from patients without (Chart A) and with (Chart C) CRC. A cluster primarily including PRX4, SDC1, VEGFA, ID1 and CRP genes define the main change in the hepatic transcriptional structure between patients without and with CRC, as shown in Charts B and D. This analysis may be automated using data processing device or equipment. Thus, according to another aspect of the invention, hierarchical clustering of PRX4, SDC1, VEGFA, ID1 and CRP genes may form a subclinical parameter or indicator that is utilized by a data processing device to systematically automate prediction of CRC cancer risk and provide an alert of possible occult CRC in patients without clinical evidence of CRC but with other diseases that increase CRC risk.

Spearman's correlation analysis was used to study the structure of transcriptional associations among liver prometastatic genes in patients with and without CRC, and to identify those gene correlations changing between patients with and without CRC. As shown in FIG. 12, correlations among genes from patients with CRC were strengthened in the metabolic bioprotection gene group, while they were lost among genes in the immune-regulation gene group. A hierarchical clustering (FIG. 13) was performed based on Pearson's correlation Euclidean distances among liver prometastatic genes and their gene dusters, and represented as dendrogram plots in order to define the transcriptional structure of prometastatic genes in hepatic biopsies from patients with and without CRC (FIG. 13, Charts A and C). A cluster including PRX4, SDC1, VEGFA, ID1 and CRP genes defined the main change in the hepatic transcriptional structure between patients with and without CRC (Charts C and D). Therefore, an additional feature contributing to identifying CRC-dependent gene expression changes in patients without clinical evidence of CRC is the correlation pattern among liver prometastatic genes.

It was also revealed that the relationship between and among gene expression levels within functional categories differ according to location of the primary tumor in patients having CRC cancer. According to another aspect of the present invention, this information may be used to determine or direct a type of treatment administered to a patient. FIGS. 4A, 4B, 4C and 4D, for example, show differences in distributions of liver prometastatic genes in high-expressing patients according to whether the primary tumor is located in the rectum (dotted trace), right-side (dot-dash trace) colon and left-side (dashed trace) colon. In particular, FIG. 4A shows a first relational distribution of liver prometastatic gene expressions in high-expressing patients for Proinflammatory genes (IL18, ID1, TNF, VEFA, EPHA1, TSFSF14, CYP2E1 and ADH1B) according to primary tumor location in patients with CRC. FIG. 4B shows a second relational distribution of liver prometastatic gene expressions in high-expressing patients for Immune regulation genes (ICAM1, IL10, MRC1, KNG1, SDI1, COL18A1, IGF1 and MP 7) according to primary tumor location in patients with CRC. FIG. 4C shows a third relational distribution of liver prometastatic gene expressions in high-expressing patients for Metabolic Bioprotection genes (GAPDH, PRDX4, TXN, MT1E, HP, NOS2, CRP, and ERBB2IP) according to primary tumor location in patients with CRC. FIG. 4D shows a fourth relational distribution of liver prometastatic gene expressions in high-expressing patients for Fibrogenic/Regeneration genes (RPL23, DDR2, TGFB1, VTN and NGF) according to primary tumor location in patients with CRC.

A further aspect of the invention includes a complementary diagnostic test to indicate a possible anatomical location of an occult CRC in patients without clinical evidence of CRC, but with other digestive system diseases increasing CRC risk, such as cholelithiasis and metabolic syndrome. As shown in FIGS. 13 and 4A-4D, patients with tumors of localization in the left-side colonic area (including splenic flexure, descending colon, sigmoid colon or recto sigmoid junction) were the ones that most frequently increased the expression of liver prometastatic genes, followed by patients with right-sided tumors (including cecum, ascending colon, hepatic flexure or transverse colon), whereas patients with rectal tumors were those more frequently decreasing the expression level. The anatomical location of CRC determined the liver prometastatic gene expression pattern and the percentage of patients with high and low expression of these genes. Therefore, in accordance with this further aspect of the invention, these patterns suggest the possible anatomical location of an occult CRC in patients without clinical evidence of CRC, but with other digestive system diseases increasing CRC risk, such as cholelithiasis and metabolic syndrome in order to provide a basis to direct and determine a best possible treatment regime

Table 6 shows distribution of liver prometastatic genes by functional categories and tumor location. Rectal Tumor Pattern is indicated by Low hepatic expression of genes from the four prometastatic gene functional categories with high-IL10, MRC1 and NOS2 gene expression, which suggest Immunotolerance/immunosuppression without inflammatory background and possible beneficial effects of immunotherapy in metastasis prevention. Left-sided colonic Tumor Pattern (including CRC within the splenic flexure, descending colon, sigmoid colon or recto sigmoid junction) is indicated by High hepatic expression of proinflammatory, immune regulation and metabolic bioprotection genes, with drop in BMP7 and NGF gene expression, which suggests very high-risk prometastatic microenvironment and possible beneficial effects of anti-inflammatory therapies in metastasis prevention. Right-sided colonic Tumor Pattern (including primary CRC in the cecum, ascending colon, hepatic flexure or transverse colon) is indicated by a slight increase of proinflammatory and immune regulation gene expression with ADH1B, SDC1 and VT gene expression decrease, which suggests slight immunotolerance/immunosuppression under inflammatory conditions and possible beneficial effect of anti-inflammatory therapies in metastasis prevention. According to yet another aspect of the present invention, an analytical determination may be made to determine a treatment regime in accordance with high-low gene expression levels of genes within respective functional categories and anatomic location of the tumor along the colonic tract. A processing device also may be utilized to provide such determination in an automated diagnostic and treatment system.

Personalized treatment of patients may be made based on a multiplex of molecular biomarkers defining precise functional features of cancer that may strongly increase the efficacy of the chosen therapies. In this study, the analysis of liver prometastatic gene functional categories by anatomical location of the CRC identified three distinct functional patterns with therapeutic implications. Rectal Tumor Pattern was indicated by Low hepatic expression of genes from the four prometastatic gene functional categories with high-IL10, MRC1 and NOS2 gene expression, which suggest Immunotolerance/immunosuppression without inflammatory background and possible beneficial effects of immunotherapy in metastasis prevention. Left-sided colonic Tumor Pattern is indicated by high hepatic expression of proinflammatory, immune regulation and metabolic bioprotection genes, with drop in BMP7 and NGF gene expression, which suggests very high-risk prometastatic microenvironment and possible beneficial effects of anti-inflammatory therapies in metastasis prevention. Right-sided colonic Tumor Pattern was indicated by Slight increase of proinflammatory and immune regulation gene expression with ADH1B, SDC1 and VT gene expression decrease, which suggests slight Immunotolerance/immunosuppression under inflammatory conditions and possible beneficial effect of anti-inflammatory therapies in metastasis prevention.

The written description, drawing figures, tables and charts presented herein are not intended to limit the scope of the invention but merely provide an illustration of the core concepts and embodiments that may be implemented to carry out the teachings set forth herein. Based on these teachings, persons skilled in the art may devise alternative embodiments or modify the illustrated embodiments without departing from the scope of the invention. Accordingly, the scope of invention is defined by the appended claims rather than by the description or illustrated embodiments.

### REFERENCES

Anasagasti M J, Alvarez A, Mendoza L, Martin J J y Vidal-Vanaclocha F. Sinusoidal endothelium release of hydrogen peroxide enhances VLA-4 mediated melanoma cell adherence and tumor cytotoxicity during interleukin-1 promotion of melanoma hepatic metastasis. Hepatology 25:840-846 (1997).

Anasagasti M J, Martin J J, Mendoza L, Obrador E, Estrela J M, McCuskey R S, and Vidal-Vanaclocha F. Glutathione protects metastatic melanoma cells against oxidative stress in the murine hepatic microvasculature. Hepatology 27: 1249-1256 (1998).

Anasagasti M J, Olaso E, Calvo F, Martin J J, Mendoza M, Bidaurrazaga J, and Vidal-Vanaclocha F. Interleukin-1 (IL-1)-Dependent and -Independent Mouse Melanoma Metastases. J Natl Cancer Institute 89:645-651 (1997).

Aoyama T, Kashiwabara K, Oba K, Honda M, Sadahiro S, Hamada C, Maeda H, Mayanagi S, Kanda M, Sakamoto J, Saji S, Yoshikawa T. Clinical impact of tumor location on the colon cancer survival and recurrence: analyses of pooled data from three large phase III randomized clinical trials. Cancer Med. 2017 Sep. 25. doi: 10.1002/cam4.1208.

Arteta B, Lasuen N, Sveinbjomssøn B, Smedsrød B and Vidal-Vanaclocha F. Murine Colon Carcinoma Cell interaction with Liver Sinusoidal Endothelium Inhibits Anti-Tumor Immunity via IL-1 Induced Mannose Receptor. Hepatology 2010; 51: 2172-2182.

Arteta B, Lasuen N, Sveinbjomssøn B, Smedsrød B and Vidal-Vanaclocha F. Murine Colon Carcinoma Cell interaction with Liver Sinusoidal Endothelium Inhibits Anti-Tumor Immunity via IL-1 Induced Mannose Receptor. Hepatology 2010; 51: 2172-2182.

Badiola I, Olaso E, Crende O, Friedman S L, Vidal-Vanaclocha F. Discoidin domain receptor 2 deficiency predisposes hepatic tissue to colon carcinoma metastasis. Gut. 2012 October;61(10):1465-72.

Badiola I, Olaso E, Crende O, Friedman S L, Vidal-Vanaclocha F. Discoidin domain receptor 2 deficiency predisposes hepatic tissue to colon carcinoma metastasis. Gut. 2012 October;61(10):1465-72.

Barbois S, Arvieux C, Leroy V, Reche F, Stürm N, Borel A L. Benefit-risk of intraoperative liver biopsy during bariatric surgery: review and perspectives. Surg Obes Relat Dis. 2017 Aug 14. pii: S1550-7289(17)30359-3.

Beaskoetxea J; Ruiz-Casares E; Telleria N; del Villar A; Garcia de Durango C; Lapuente F; Gil A; Fernandez-Nespral V; Ielpo B; Carusso R; Duran H; Quijano Y; de Vicente E; Vidal-Vanaclocha F. Liver metastasis-Associated colon cancer cell genes: microenvironmental regulation and therapeutic implications. J Hepatology (in preparation).

Carrascal T, Mendoza L, Vacarcel M, Salado C, Egilegor E, Telleria N, Vidal-Vanaclocha F and Dinarello C A. Interleukin/18 binding protein reduces B16 Melanoma Hepatic Metastasis by neutralizing the adhesiveness and growth factors of sinusoidal endothelial cell. Cancer Res 63:491-7 (2003)

Crende O, Sabatino M, Valcárcel M, Carrascal T, Riestra P, López-Guerrero J A, Nagore E, Mandruzzato S, Wang E, Marincola F M, Vidal-Vanaclocha F. Metastatic lesions with and without interleukin-18-dependent genes in advanced-stage melanoma patients. Am J Pathol. 2013 July;183(1):69-82.

Garcia de Durango C, Marina Pérez-Gordo; Eva Ruiz Casares, and Fernando Vidal-Vanaclocha. Transcriptional Association of Bacterial Endotoxin-Dependent Colorectal Cancer Cell Soluble Proteins to Hepatic Prometastatic Signature Genes. Gastroenterology (in preparation).

Garcia De Durango Cr, De Wit M, Piersma Sr, Knol J, Pham Tv, Pérez-Gordo M, Fijneman Rja, Vidal-Vanaclocha F, Jimenez Cr. Lipopolysaccharide-regulated secretion of soluble and vesicle-based colorectal cancer cell proteins. J Proteomics 2017 (in preparation).

Gil A; Garcia de Durango C; Lapuente F; Fernandez-Nespral V; Ielpo B; Carusso R; Duran H; Quijano Y; de Vicente E; Vidal-Vanaclocha F. Pathophysiological anthropometric correlations of liver prometastatic genes in patients with primary colorectal cancer. Am J Pathol (in preparation).

Gosavi S, Mishra R R, Kumar V P. Study on the Relation between Colorectal Cancer and Gall Bladder Disease. J Clin Diagn Res. 2017 March;11(3):OC25-OC27.

Lee T, Yun K E, Chang Y, Ryu S, Park D I, Choi K, Jung Y S. Risk of Colorectal Neoplasia According to Fatty Liver Severity and Presence of Gall Bladder Polyps. Dig Dis Sci. 2016 January;61(1):317-24.

Márquez J, Kohli M, Arteta B, Chang S, Li W B, Goldblatt M, Vidal-Vanaclocha F. Identification of hepatic microvascular adhesion-related genes of human colon cancer cells using random homozygous gene perturbation. Int J Cancer. 2013 November;133(9):2113-22.

Márquez J, Kohli M, Arteta B, Chang S, Li W B, Goldblatt M, Vidal-Vanaclocha F. Identification of hepatic microvascular adhesion-related genes of human colon cancer cells using random homozygous gene perturbation. Int J Cancer. 2013 November;133(9):2113-22.

Marshall J C, Collins J W, Nakayama J, Horak C E, Liewehr D J, Steinberg S M, Albaugh M, Vidal-Vanaclocha F, Palmieri D, Barbier M, Murone M, Steeg P S. Effect of inhibition of the lysophosphatidic acid receptor 1 on metastasis and metastatic dormancy in breast cancer. J Natl Cancer Inst. 2012 Sep. 5;104(17):1306-19.

Mendoza L, Carrascal T, De Luca M, Fuentes A M, Salado C, Blanco J, Vidal-Vanaclocha F. Hydrogen peroxide mediates vascular cell adhesion molecule-1 expression from interleukin-18-activated hepatic sinusoidal endothelium: implications for circulating cancer cell arrest in the murine liver. Hepatology 34:298-310 (2001).

Mendoza L, Valcarcel M, Carrascal T, Egilegor E, Salado C, Sim B K, Vidal-Vanaclocha F. Inhibition of cytokine-induced microvascular arrest of tumor cells by recombinant endostatin prevents experimental hepatic melanoma metastasis. Cancer Res 64: 304-10 (2004)

Olaso E, Salado C, Gutierrez V, and Vidal-Vanaclocha F. Proangiogenic Role of Tumor-Activated Hepatic Stellate Cells in Melanoma Metastasis. Hepatology 37:674-85 (2003)

Olaso E, Santisteban A, Bidaurrazaga J, Gressner A M, Rosenbaum J, and Vidal-Vanaclocha F. Tumor-dependent activation of hepatic stellate cells during experimental melanoma metastasis. Hepatology 26: 634-642 (1997).

Ruiz-Casares E; Lapuente F; Ielpo B; Carusso R; Duran H; Quijano Y; de Vicente E; Vidal-Vanaclocha F. Prometastatic gene expression patterns in the liver of patients with and without colorectal cancer: pathogenic implications and clinical correlations. Hepatology (in preparation)

Solaun M S, Mendoza L, de Luca M, Gutierrez V, López M-P, Olaso E, B, Sim B K L, Vidal-Vanaclocha F. Endostatin Inhibits Murine Colon Carcinoma Sinusoidal-Type Metastases by Preferential Targeting of Hepatic Sinusoidal Endothelium. Hepatology 35: 1104-1116 (2002)

Valcárcel M, Carrascal T, Crende O, Vidal-Vanaclocha F. IL-18 Regulates Melanoma VLA-4 Integrin Activation through a Hierarchized Sequence of Inflammatory Factors. J Invest Dermatol. 2014 February 134(2):470-80.

Vidal-Vanaclocha F, Fantuzzi G, Mendoza L, Fuentes A M, Anasagasti M J, Martin J J, Carrascal T, Walsh P, Reznikov L L, Kim S-H, Novick D, Rubinstein M, and Dinarello C A. IL-18 regulates IL-1beta-dependent hepatic melanoma metastasis via vascular cell adhesion molecule-1. Proc Nat Acad Sci USA 97: 734-39 (2000).

Vidal-Vanaclocha F, Mendoza L, Telleria N, Salado C, Valcarcel M, Gallot N, Carrascal T, Egilegor E, Beaskoetxea J, Dinarello. Clinical and experimental approaches to the pathophysiology of interleukin-18 in cancer progression. Cancer Metastasis Rev 25:417-34 (2006)

Vidal-Vanaclocha F. Architectural and Functional Aspects of the Liver with Implications for Cancer Metastasis. P. Brodt (ed.), Liver Metastasis: Biology and Clinical Management, Cancer Metastasis—Biology and Treatment 16, DOI 10.1007/978-94-007-0292-9_2, C_Springer Science+Business Media B.V. 2011a.

Vidal-Vanaclocha F. Regulation of Liver Metastasis-Related Genes at Primary and Metastatic Tumors in the Pathophysiological Context of the Colorectal Cancer Disease. Gut (review article in preparation).

Vidal-Vanaclocha F. The Liver Prometastatic Reaction of Cancer Patients: Implications for Microenvironment-Dependent Colon Cancer Gene Regulation. CAMI 2011;4(2):163-80.

Vidal-Vanaclocha F. The prometastatic microenvironment of the liver. Cancer Microenvir, 2008; 1: 113-129

Vidal-Vanaclocha F. The Tumor Microenvironment at Different Stages of Hepatic Metastasis P. Brodt (ed.), Liver Metastasis: Biology and Clinical Management, Cancer Metastasis—Biology and Treatment 16, DOI 10.1007/978-94-007-0292-9_3, C_Springer Science+Business Media B.V. 2011 b.

Yahagi M, Okabayashi K, Hasegawa H, Tsuruta M, Kitagawa Y. The Worse Prognosis of Right-Sided Compared with Left-Sided Colon Cancers: a Systematic Review and Meta-analysis. J Gastrointest Surg. 2016 March;20(3):648-55.

Zhao X, Li L, Starr T K, Subramanian S. Tumor location impacts immune response in mouse models of colon cancer. Oncotarget. 2017 Jun. 9;8(33):54775-54787.

I claim:

1. A method of detecting metastatic cancer in a target patient having a colorectal tumor, said method comprising:
(a) obtaining a hepatic tissue sample from the target patient;
(b) measuring genetic expression levels of a number of genes in said tissue sample selected from group 1 (PRDX4, CRP, ID1, MT1E, TNFSF14, MRC1, ICAM1, IL18, IL10, TFN) and/or group 2 (NGF, EPHA1, ERBB2IP, SDC1, COL18A1, KNG1, ADH1B, CYP2E1);
(c) comparing expression levels of genes measured in said group 1 and/or group 2 with expression levels of respective genes indicative of a person free of a colorectal tumor; and
(d) detecting that the target patient has metastatic cancer if certain ones of group 1 genes are overexpressed and/or certain ones of group 2 genes are underexpressed.

2. The method of claim 1, wherein the detecting step is carried out by detecting if a statistically significant number of group 1 and/or group 2 genes are respectively overexpressed or underexpressed.

3. The method of claim 1, wherein the detecting step is carried out by detecting if statistically significant ones of genes of group 1 and/or group 2 genes are respectively overexpressed and/or underexpressed.

4. The method of claim 1, wherein said measuring step further includes:
measuring genetic expression levels of genes of said tissue sample selected from group 3 genes (HP, VTN, RPS27, RPL23, GAPDH, TXN, VEGFA, CEACAM1, IGF1, TGFB1, DDR2, NOS2, and BMP7) and
detecting that expression levels of group 3 genes are congruent both for patients with and without CRC in order to validate the expression levels of group 1 and group 2 genes.

5. The method of claim 4, wherein said comparing step further includes:
examining correlation, clustering patterns and/or partial least squares discriminatory analysis of gene expression levels measured in said group 1, group 2 and/or group 3 genes relative to expression levels of genes indicative of individuals free of colorectal tumors.

6. The method of claim 1, where said detecting step further includes:
in comparison with corresponding genes of persons free of CRC, detecting (i) overexpression of selected ones of PRDX4, MT1E, TNFSF14, MRC1, ICAM1, IL18, IL10, TNF, ID1 and CRP genes; (ii) underexpression of selected ones of NGF, EPHA1, ERBB2IP, SDC1, COL18A1, KNG1, ALDH1B, CYP2E1 genes; (iii) altered correlation patterns of expressions among metabolic bioprotection genes and among proinflammatory and metabolic bioprotection genes; (iv) loss of expression correlation among proinflammatory-fibrogenic/regeneration and immune regulation genes; or (v) new gene clustering patterns for PRDX4, SDC1, VEGFA, ID1 and CRP genes.

7. A method of detecting occult cancer in a target patient having a gastrointestinal disorder, said method comprising:
(a) obtaining a hepatic tissue sample from said patient;
(b) in said hepatic tissue sample, measuring expression levels of statistically significant ones of (i) metabolic bioprotection genes PRDX4, MT1E, CRP and NOS2, (ii) immune regulation genes ICAM1, IL10 and MRC1, or (iii) proinflammatory genes ID1, TNF-a, IL18 and TNFSF14 and/or statistically significant ones of (i) immune-regulation genes SDC1, COL18A1 and KNG1, (ii) proinflammatory genes EPHA1, CYP2E1, ADH1 B, or (iii) fibrogenic/regeneration gene NGF; and
(c) detecting occult cancer in said target patient if there are increased expression levels of statistically significant ones of (i) metabolic bioprotection genes PRDX4, MT1E, CRP and NOS2, (ii) immune regulation genes ICAM1, IL10 and MRC1, or (iii) proinflammatory genes ID1, TNF-a, IL18 and TNFSF14 and/or decreased expression levels of statistically significant ones of (i) immune-regulation genes SDC1, COL18A1 and KNG1, (ii) proinflammatory genes EPHA1, CYP2E1, ADH1 B, or (iii) fibrogenic/regeneration gene NGF.

8. The method of claim 7, wherein said detecting step further includes:
comparing expressions levels to detect (i) new correlations of expression levels among metabolic bioprotection genes and among proinflammatory and metabolic bioprotection genes and/or (ii) hierarchal clustering to detecting express/reaction levels of PRX4, SDC1, VEGFA, ID1 and CRP genes; and/or (iii) loss of expression correlation among proinflammatory-fibrogenic/regeneration and immune regulation genes wherein new clustering patterns or lost correlation for PRDX4, SDC1, VEGFA, ID1 and CRP genes indicate occult CRC in patients having no previous clinical evidence of CRC.

9. A method of diagnosing and treating a patient suspected of having a subclinical liver micrometastasis disease or subclinical liver metastasis with a targeted gene therapy comprising, said method comprising:
(a) obtaining a hepatic tissue sample;
(b) measuring in said hepatic tissue sample expression levels of genes from statistically significant ones of group 1 genes (PRDX4, CRP, ID1, MT1E, TNFSF14, MRC1, ICAM1, IL18, IL10, TFN), group 2 genes (NGF, EPHA1, ERBB2IP, SDC1, COL18A1, KNG1, ADH1 B, CYP2E1) and/or group 3 genes (HP, VTN, RPS27, RPL23, GAPDH, TXN, VEGFA, CEACAM1, IGF1, TGFB1, DDR2, NOS2, and BMP7);
(c) comparing said measured expressions levels of said genes in the previous step with expression levels of said corresponding genes of persons known to be free of colorectal cancer;
(d) identifying over-expressed and under-expressed gene expressions according to proinflammatory, immune regulation, metabolic protection and fibrogenic/regeneration classes of genes; and
(e) treating said patient with anti-inflammatory therapy or immunotherapy according extent of over-expressed and under-expressed genes residing in said respective classes.

10. The method of claim 9, further comprising treating a rectal tumor of said patient using immunotherapy according to a high expression level of IL10, MRC1 and NOS2 genes.

11. The method of claim 9, further comprising treating a left-sided colonic tumor of said patient using an anti-inflammatory therapy according to high expressions of proinflammatory, immune regulation and metabolic bioprotection genes and decreased expression of BMP7 and NGF genes.

12. The method of claim 9, further comprising treating a right-sided colonic tumor of said patient using anti-inflammatory therapy according to indication of a slight increase of proinflammatory and immune regulation gene expressions and decrease in ADH1B, SDC1 and VT gene expressions.

13. The method of claim 9, further comprising treating said patient by administering a drug that targets selected liver prometastatic genes, as well as gene expression products and receptors thereof and associated signaling pathways thereof.

14. A method of detecting anatomical location of an occult CRC tumor in a patient without clinical evidence of CRC, said method comprising;
(a) obtaining a hepatic biopsy,
(b) measuring in said biopsy expression levels of selected ones of prometastatic genes within proinflammatory, immune regulation, bioprotection and fibrogenic/regeneration functional classes of genes from selected ones of group 1 genes PRDX4, CRP, ID1, MT1E, TNFSF14, MRC1, ICAM1, IL18, IL10, TFN), group 2 genes (NGF, EPHA1, ERBB2IP, SDC1, COL18A1, KNG1, ADH1B, CYP2E1) and/or group 3 genes (HP, VTN, RPS27, RPL23, GAPDH, TXN, VEGFA, CEACAM1, IGF1, TGFB1, DDR2, NOS2, and BMP7;
(c) determining the identity of over-expressed and under-expressed ones of said selected prometastatic genes within said respective classes of genes, and
(d) detecting said anatomical location of said occult CRC in said patient according identified ones of over-expressed and under-expressed genes residing in said respective classes.

15. The method of claim 14, wherein said detecting step comprises detecting a rectal location of said CRC tumor according to underexpressed levels of statistically significant ones of (i) IL18, ID1, VEGFA, TNFSF14, ADH1B and CYP2E1 proinflammatory genes, (ii) ICAM1, KNG1, SDC1 AND BMP7 immuno regulation genes, and (iii) GAPDH, TXN, MTE1, HP, CR AND ERBB2IP metabolic bioprotection genes.

16. The method of claim 14, where said detecting step comprises detecting a left-side colon location of said CRC tumor according to overexpressed levels of statistically significant ones of (i) proinflammatory genes IL18, ID1, TNF, TNFSF14, AND ADH1B, (ii) immune regulation genes ICAM1, MRC1, KNG1, and SDC1, and/or (iii) metabolic bioprotection genes PRXD4, MTE1, P, NOS2 and CRP.

17. The method of claim 14, where said detecting step comprises detecting right side colon location of said CRC tumor according to (i) high expression level of at least one of ID1 and TNF proinflammatory genes, (ii) low expression level of at least one of ADH18 and CYPE1 proinflammatory genes, (iii) high expression level of at least one of immune regulation genes IL10, MRC1 and BMP7, (iv) low expression level of at least one of immune regulation genes KNG1 and SDC1, and (v) low expression level of at least one of VTN and NGF fibrogenic and regeneration genes.

18. A method of diagnosing and treating a patient with CRC comprising (a) obtaining from the patient a sample of hepatic tissue or blood serum/plasma, (b) in said sample, measuring expression levels of liver prometastatic genes or proteins to identify abnormal genes or gene products (i.e., protein production) being overexpressed and/or underexpressed, and (c) treating the patient with a liver metastasis-specific therapy that targets (i) said abnormal genes, (ii) specific gene expression products or receptors of said abnormal genes and/or (iii) associated signaling pathways of said abnormal genes.

19. A method of detecting liver metastasis or risk thereof in a patient afflicted with CRC, obesity, gallstones, or other disease increasing CRC risk, said method comprising the steps of (a) obtaining from the patient a sample of blood serum or plasma to be examined; (b) determining a protein signature of the sample by measuring the presence and/or amount of two or more proteins encoded by the genes of group 1 genes (PRDX4, CRP, ID1, MT1E, TNFSF14, MRC1, ICAM1, IL18, IL10, TFN) and/or group 2 genes (NGF, EPHA1, ERBB2IP, SDC1, COL18A1, KNG1, ADH1B, CYP2E1), and (c) detecting liver metastasis if the presence and/or amount of the two or more proteins differs from a baseline protein signature of a normal, or healthy, individual not suffering from CRC, obesity, gallstones, or other gastrointestinal disease.

20. The method of claim 18, further comprising a method according to the preceding steps to detect beneficial effects of treatment of the patient, further comprising a step (d) of repeating steps (a), (b) and (c) to assess reduction in differences between said protein signatures whereby to indicate of treatment with said therapeutic agent has a beneficial effect.
